(19) United States
(12) Patent Application Publication
(10) Pub. No.: US 2019/0100745 A1
(43) Pub. Date: Apr. 4, 2019

(54) NOVEL CAS SYSTEMS AND METHODS OF USE

(71) Applicant: PIONEER HI-BRED INTERNATIONAL, INC., Johnston, IA (US)

(72) Inventors: ANDREW MARK CIGAN, MADISON, WI (US); ZHENGLIN HOU, ANKENY, IA (US); MATTHEW G. KING, JOHNSTON, IA (US); HAINING LIN, CLIVE, IA (US); JOSHUA K. YOUNG, JOHNSTON, IA (US)

(73) Assignee: PIONEER HI-BRED INTERNATIONAL, INC., JOHNSTON, IA (US)

(21) Appl. No.: 16/099,765

(22) PCT Filed: Jun. 1, 2017
(86) PCT No.: PCT/US17/35425

§ 371 Date: Nov. 8, 2018

Related U.S. Application Data
(60) Provisional application No. 62/352,193, filed on Jun. 20, 2016.
(60) Provisional application No. 62/435,145, filed on Dec. 16, 2016.

(51) Int. Cl. C12N 15/10; C12N 15/82; C12N 15/113; C12N 9/22

(52) U.S. Cl. CPC C12N 15/102; C12N 15/8213; C12N 2310/20; C12N 9/22; C12Y 301/00; C12N 15/113

(57) ABSTRACT

Compositions and methods are provided for genome modification of a target sequence in the genome of a cell. The methods and compositions employ a guide polynucleotide/Cas endonuclease system to provide an effective system for modifying or altering target sequences within the genome of a cell or organism. Also provided are Cas endonucleases comprising previously undefined nuclease domains and methods employing said Cas endonucleases for production of a guide polynucleotide/Cas endonuclease systems, for genome editing of a nucleotide sequence in the genome of a prokaryotic or eukaryotic cell, and/or for inserting or deleting a polynucleotide of interest into or from the genome of an organism.

### CROSS-REFERENCE TO RELATED APPLICATIONS

This application is a 371 National Stage Entry of PCT Application No. PCT/US17/35425 filed on 1 Jun. 2017, which claims the benefit of U.S. Provisional Application No. 62/352,193, filed Jun. 20, 2016, and U.S. Provisional Application No. 62/435,145, filed Dec. 16, 2016, all of which are hereby incorporated herein in their entirety by reference.

### FIELD

The disclosure relates to the field of plant molecular biology, in particular, to, to compositions of guide polynucleotide/Cas endonuclease systems and compositions and methods for altering the genome of a cell.

### REFERENCE TO SEQUENCE LISTING SUBMITTED ELECTRONICALLY

The official copy of the sequence listing is submitted electronically via EFS-Web as an ASCII formatted sequence listing with a file named 20170522_7129PCT_SequenceListing_ST25.txt, created May 22, 2017 and having a size of 923 kilobytes and is filed concurrently with the specification. The sequence listing contained in this ASCII formatted document is part of the specification and is herein incorporated by reference in its entirety.

### BACKGROUND

Recombinant DNA technology has made it possible to insert DNA sequences at targeted genomic locations and/or modify specific endogenous chromosomal sequences. Site-specific integration techniques, which employ site-specific recombination systems, as well as other types of recombination technologies, have been used to generate targeted insertions of genes of interest in a variety of organism. Genome-editing techniques such as designer zinc finger nucleases (ZFNs) or transcription activator-like effector nucleases (TALENs), or homing meganucleases, are available for producing targeted genome perturbations, but these systems tends to have a low specificity and employ designed nucleases that need to be redesigned for each target site, which renders them costly and time-consuming to prepare.

Although several approaches have been developed to target a specific site for modification in the genome of a prokaryotic or eukaryotic organism, there still remains a need for more effective genome engineering technologies that are affordable, easy to set up, scalable, and amenable to targeting multiple positions within the plant genome.

### BRIEF SUMMARY

Compositions and methods are provided for novel Cas systems and elements comprising such systems, including, but not limited, novel guide polynucleotide/Cas endonuclease complexes, guide polynucleotides, guide RNA elements, and Cas endonucleases, in particular, to Cas endonucleases comprising previously undefined nuclease domains. Compositions and methods are also provided for direct delivery of Cas endonucleases comprising previously undefined nuclease domains, chimeric engineered guide RNAs and guide RNA/Cas endonucleases complexes, as well as for genome modification of a target sequence in the genome of a prokaryotic or eukaryotic cell, and/or for inserting or deleting a polynucleotide of interest into or from the genome of an organism.

In one embodiment of the disclosure, the guide polynucleotide/Cas endonuclease complex comprises at least one chimeric engineered guide RNA and a Cas endonuclease, where the Cas endonuclease comprises a first nuclease domain of SEQ ID NO: 88 or a functional fragment of SEQ ID NO: 88, and a second nuclease domain comprising at least one nuclease subdomain selected from the group consisting of SEQ ID NO: 90, SEQ ID NO: 92 and SEQ ID NO: 94, or a functional fragment of the second domain, where the guide RNA/Cas endonuclease complex is capable of recognizing, binding to, and optionally nicking, cleaving, or covalently attaching to all or part of a target sequence.

In one embodiment of the disclosure, the guide polynucleotide is a chimeric engineered guide RNA capable of forming a guide RNA/Cas endonuclease complex with a Lapis Cas endonuclease, so that the complex can recognize, bind to, and optionally nick, cleave, or covalently attach to a target sequence, where the chimeric engineered guide RNA is selected from the group consisting of SEQ ID NOs: 128-138.

In one embodiment of the disclosure, the method comprises a method for modifying a target site in the genome of a cell, comprising introducing into the cell at least one chimeric engineered guide RNA, and a Cas endonuclease comprising a first nuclease domain of SEQ ID NO: 88, and a second nuclease domain comprising at least one nuclease subdomain selected from the group consisting of SEQ ID NO: 90, SEQ ID NO: 92 and SEQ ID NO: 94, where the chimeric engineered guide RNA and Cas endonuclease can form a complex that is capable of recognizing, binding to, and optionally nicking, cleaving, or covalently attaching to all or part of the target site.

In one embodiment of the disclosure, the method comprises a method for for editing a nucleotide sequence in the genome of a cell, the method comprising introducing into the cell at least one polynucleotide modification template, at least one chimeric engineered guide RNA, and a Cas endonuclease comprising a first nuclease domain of SEQ ID NO: 88, and a second nuclease domain comprising at least one nuclease subdomain selected from the group consisting of SEQ ID NO: 90, SEQ ID NO: 92 and SEQ ID NO: 94, where the polynucleotide modification template comprises at least one nucleotide modification of the nucleotide sequence, where the chimeric engineered guide RNA and Cas endonuclease can form a complex that is capable of recognizing, binding to, and optionally nicking, cleaving, or covalently attaching to all or part of the target site.

In one embodiment of the disclosure, the method comprises a method for modifying a target site in the genome of a cell, the method comprising providing to the cell at least one chimeric engineered guide RNA, at least one donor DNA, and a Cas endonuclease comprising a first nuclease domain of SEQ ID NO: 88, and a second nuclease domain comprising at least one nuclease subdomain selected from the group consisting of SEQ ID NO: 90, SEQ ID NO: 92 and SEQ ID NO: 94, where the at least one chimeric engineered guide RNA and Cas endonuclease can form a complex that is capable of recognizing, binding to, and optionally nicking, cleaving, or covalently attaching to all or part of the target site, where the donor DNA comprises a polynucleotide of interest. The method can further comprise identifying at least one cell that the polynucleotide of interest integrated in or near the target site.

In one embodiment of the disclosure, the recombinant DNA polynucleotide comprising a promoter operably linked to a eukaryotic-optimized polynucleotide encoding a Cas endonuclease, where the Cas endonuclease comprises a first nuclease domain of SEQ ID NO: 88, and a second nuclease domain comprising at least one nuclease sub-domain selected from the group consisting of SEQ ID NO: 90, SEQ ID NO: 92 and SEQ ID NO: 94.

In one embodiment of the disclosure, the kit comprises a kit for binding, cleaving or nicking a target sequence in a prokaryotic or eukaryotic cell or organism, the kit comprising a guide polynucleotide specific for the target sequence, and a Cas endonuclease or a polynucleotide encoding the Cas endonuclease, where the Cas endonuclease comprises a first nuclease domain of SEQ ID NO: 88, and a second nuclease domain comprising at least one nuclease sub-domain selected from the group consisting of SEQ ID NO: 90, SEQ ID NO: 92 and SEQ ID NO: 94, where the guide polynucleotide is capable of forming a guide polynucleotide/Cas endonuclease complex, where the complex can recognize, bind to, and optionally nick or cleave the target sequence.

Also provided are nucleic acid constructs, eukaryotic cells, plants, plant cells, explants, seeds and grain having a modified target sequence or having a modification at a nucleotide sequence in the genome of the plant, produced by the methods described herein. Additional embodiments of the methods and compositions of the present disclosure are shown herein.

### BRIEF DESCRIPTION OF THE DRAWINGS AND THE SEQUENCE LISTING

The disclosure can be more fully understood from the following detailed description and the accompanying drawings and Sequence Listing, which form a part of this application. The sequence descriptions and sequence listing attached hereto comply with the rules governing nucleotide and amino acid sequence disclosures in patent applications as set forth in 37 C.F.R. §§ 1.821-1.825. The sequence descriptions contain the three letter codes for amino acids as defined in 37 C.F.R. §§ 1.821-1.825, which are incorporated herein by reference.

### FIGURES

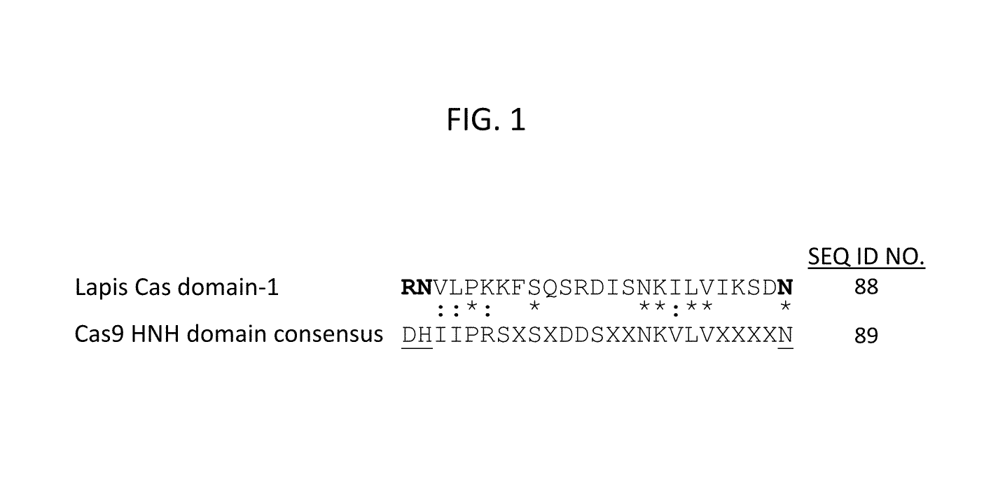

FIG. 1 depicts an alignment of a previously unidentified nuclease domain (referred to as Lapis Cas nuclease domain1; SEQ ID NO: 88) from the novel Cas protein of Lactobacillus apis (Lapis) with the HNH consensus domain from 86 diverse Cas9 proteins (SEQ ID NO: 97). Underlined residues represent the key catalytic residues of the HNH domain. Amino acid residues in bold represent the corresponding residues in the Cas protein from Lapis. An “*” denotes a perfect match with the domain consensus and a “:” indicates a conservative match with the domain consensus. The 86 diverse Cas9 proteins were aligned using MUSCLE (Edgar R. (2004) Nucleic Acids Research, 32(5): 1792-97) and a consensus calculated based on the amino acid frequency at each position. Only the most abundant amino acid at each position was reported and an “X” was used to denote the lack of a significant amino acid preference (X can be any amino acid). Additionally, positions within the domain consensus where only a few Cas9 proteins provided an alignment were omitted to reduce gaps in the final consensus.

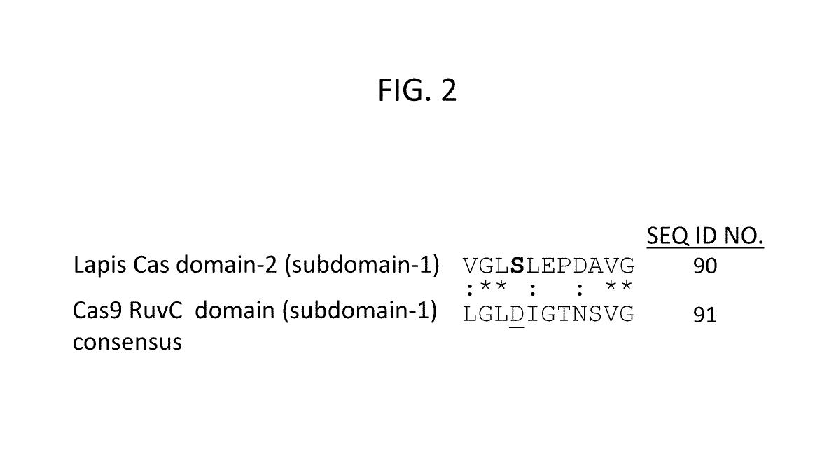

FIG. 2 depicts an alignment of a previously unidentified nuclease subdomain (referred to as Lapis Cas nuclease domain2-subdomain-1; SEQ ID NO: 90) from the novel Cas protein of Lactobacillus apis (Lapis) with the RuvC consensus domain (subdomain-1) from 86 diverse Cas9 proteins (SEQ ID NO: 91). Underlined residue represents the key catalytic residue of the RuvC subdomain. Amino acid residues in bold represent the corresponding residues in the Cas protein from Lapis. An “*” denotes a perfect match with the domain consensus and a “:” indicates a conservative match with the domain consensus. The 86 diverse Cas9 proteins were aligned using MUSCLE (Edgar R. (2004) Nucleic Acids Research, 32(5): 1792-97) and a consensus calculated based on the amino acid frequency at each position. Only the most abundant amino acid at each position was reported and an “X” was used to denote the lack of a significant amino acid preference. Additionally, positions within the domain consensus where only a few Cas9 proteins provided an alignment were omitted to reduce gaps in the final consensus.

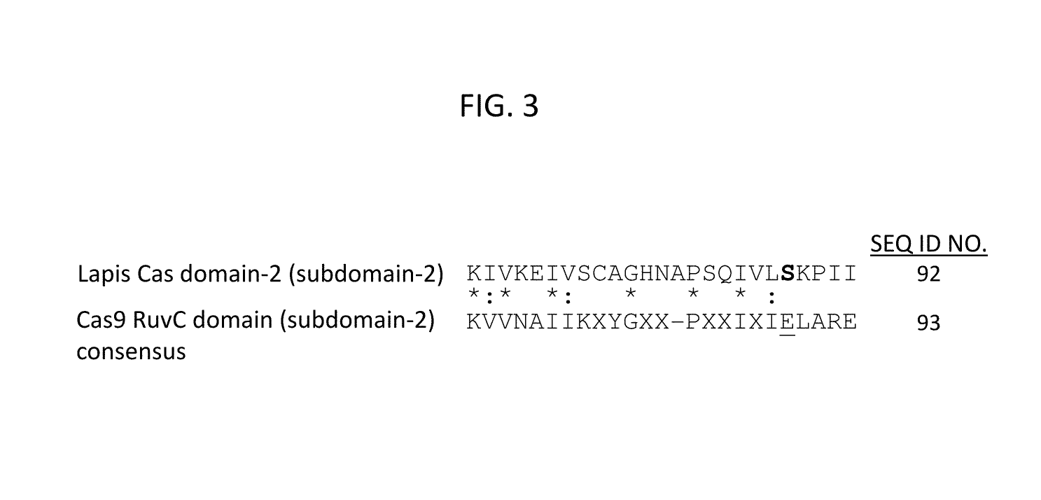

FIG. 3 depicts an alignment of a previously unidentified nuclease subdomain (referred to as Lapis Cas nuclease domain2-subdomain-2; SEQ ID NO: 92) from the novel Cas protein of Lactobacillus apis (Lapis) with the RuvC consensus domain (subdomain-2) from 86 diverse Cas9 proteins (SEQ ID NO: 93). Underlined residue represents the key catalytic residue of the RuvC subdomain. Amino acid residues in bold represent the corresponding residues in the Cas protein from Lapis. An “*” denotes a perfect match with the domain consensus, a “:” indicates a conservative match with the domain consensus and a “-” indicates a gap in the alignment. The 86 diverse Cas9 proteins were aligned using MUSCLE (Edgar R. (2004) Nucleic Acids Research, 32(5): 1792-97) and a consensus calculated based on the amino acid frequency at each position. Only the most abundant amino acid at each position was reported and an “X” was used to denote the lack of a significant amino acid preference. Additionally, positions within the domain consensus where only a few Cas9 proteins provided an alignment were omitted to reduce gaps in the final consensus.

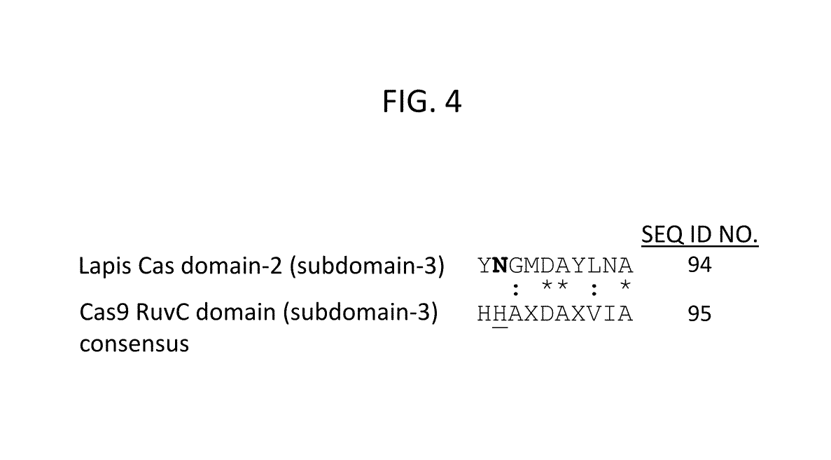

FIG. 4 depicts an alignment of a previously unidentified nuclease subdomain (referred to as Lapis Cas nuclease domain2-subdomain-3; SEQ ID NO: 94) from the novel Cas protein of Lactobacillus apis (Lapis) with the RuvC consensus domain (subdomain-3) from 86 diverse Cas9 proteins (SEQ ID NO: 95). Underlined residue represents the key catalytic residue of the RuvC subdomain. Amino acid residues in bold represent the corresponding residues in the Cas protein from Lapis. An “*” denotes a perfect match with the domain consensus and a “:” indicates a conservative match with the domain consensus. The 86 diverse Cas9 proteins were aligned using MUSCLE (Edgar R. (2004) Nucleic Acids Research, 32(5): 1792-97) and a consensus calculated based on the amino acid frequency at each position. Only the most abundant amino acid at each position was reported and an “X” was used to denote the lack of a significant amino acid preference. Additionally, positions within the domain consensus where only a few Cas9 proteins provided an alignment were omitted to reduce gaps in the final consensus.

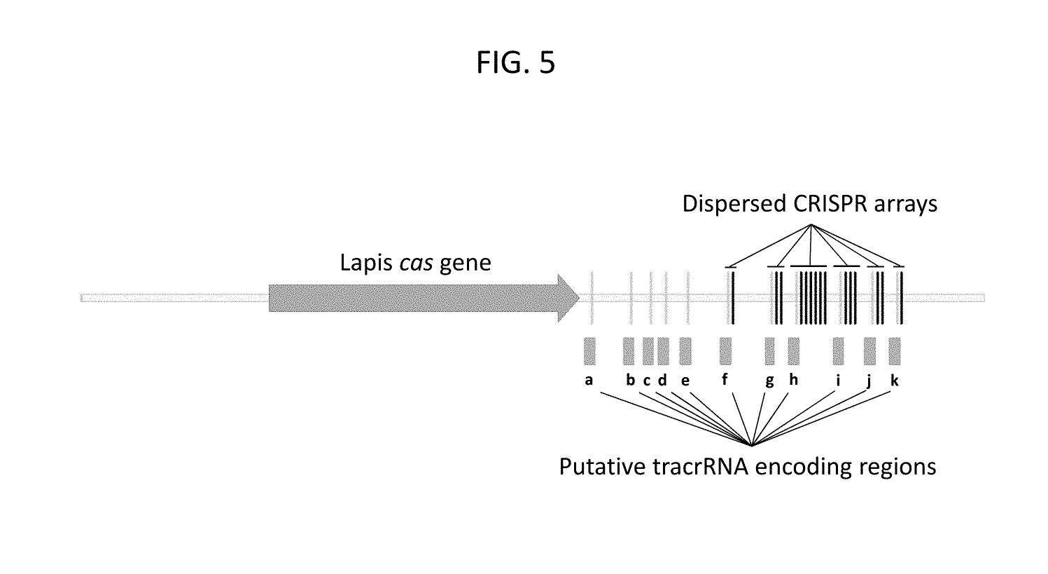

FIG. 5. Depiction of CRISPR-Cas locus structure for Lactobacillus apis (Lapis). The Lapis cas gene position and orientation relative to the CRISPR arrays is indicated. CRISPR arrays and putative tracrRNA encoding regions are labeled. Light gray lines represent regions of the putative tracrRNA with strong homology to the CRISPR repeat.

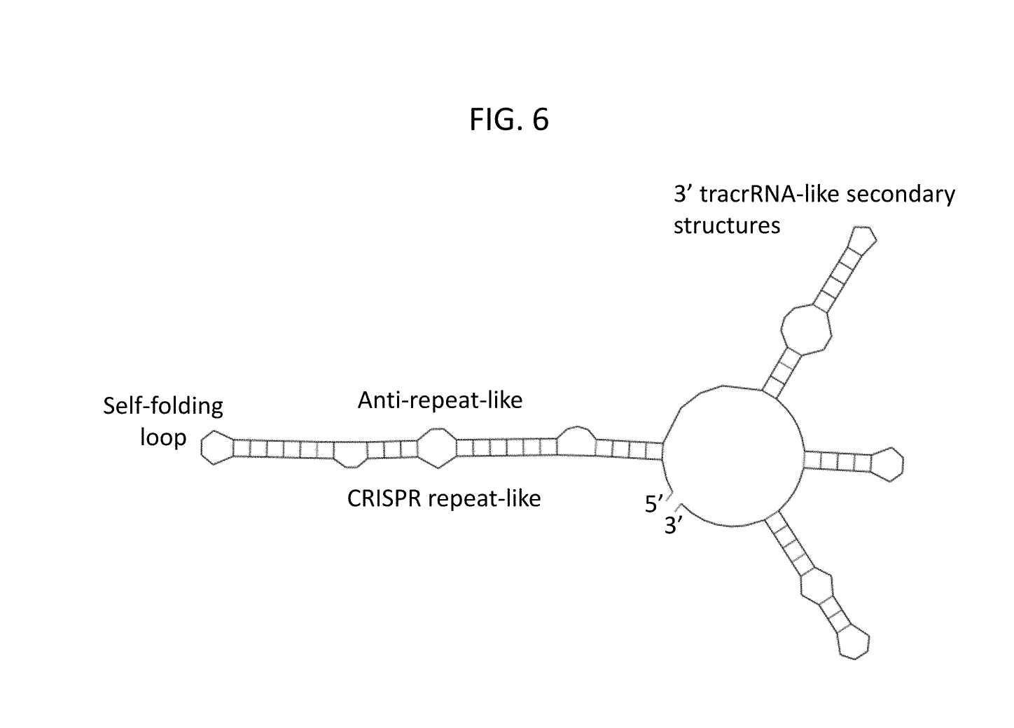

FIG. 6. Depiction of 5 prime secondary structure detected for putative tracrRNA (a) (SEQ ID NO: 99) from Lactobacillus apis (Lapis) CRISPR-Cas system when simulated to be transcribed in an anti-sense direction relative to the Lapis cas gene (SEQ ID NO: 110). RNA secondary structure was examined using UNAfold (Markham and Zuker (2008) Methods Mol Biol. 453:3-31).

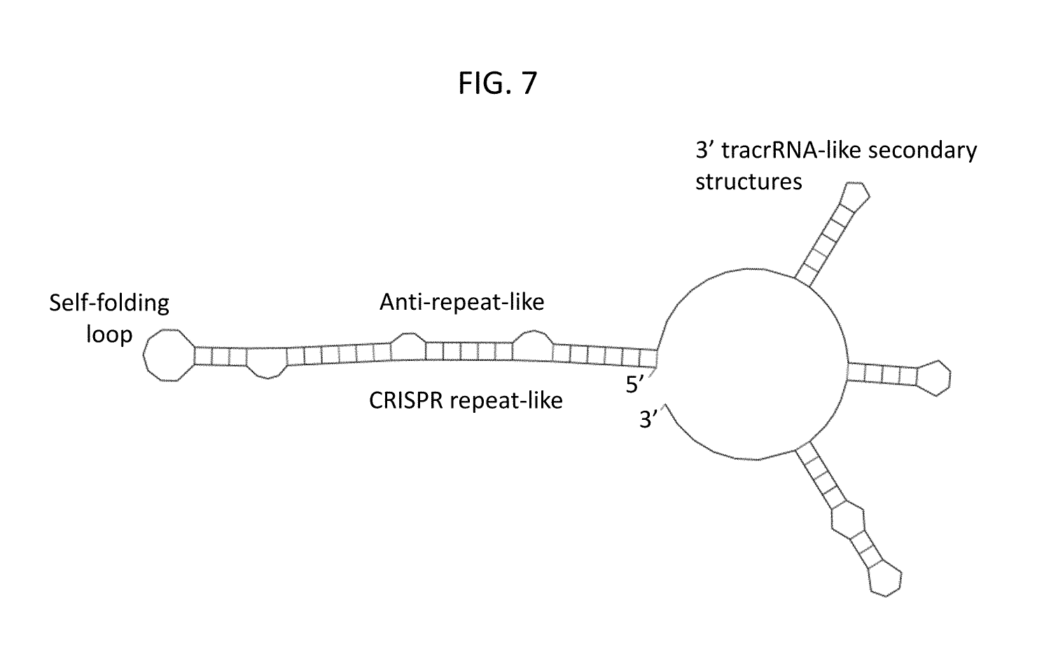

FIG. 7. Depiction of 5 prime secondary structure detected for putative tracrRNA (b) (SEQ ID NO: 100) from Lactobacillus apis (Lapis) CRISPR-Cas system when simulated to be transcribed in an anti-sense direction relative to the Lapis cas gene (SEQ ID NO: 111). RNA secondary structure was examined using UNAfold (Markham and Zuker (2008) Methods Mol Biol. 453:3-31).

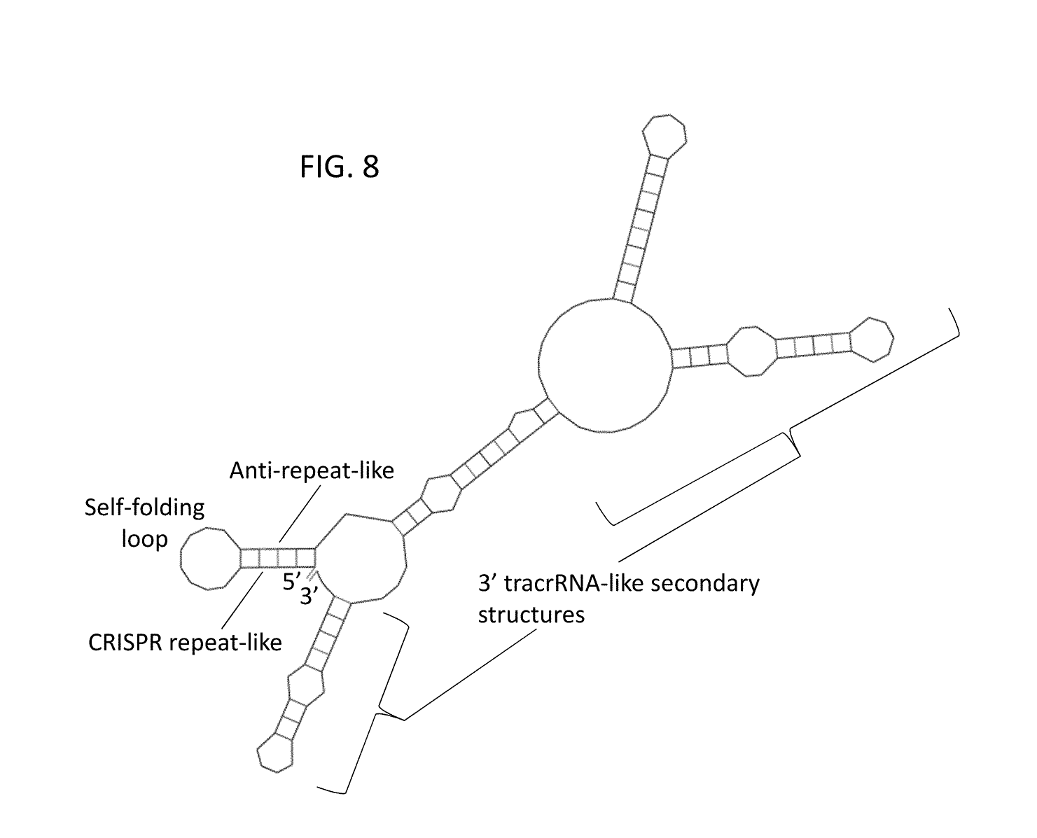

FIG. 8. Depiction of 5 prime secondary structure detected for putative tracrRNA (c) (SEQ ID NO: 101) from Lactobacillus apis (Lapis) CRISPR-Cas system when simulated to be transcribed in an anti-sense direction relative to the Lapis cas gene (SEQ ID NO: 112). RNA secondary structure was examined using UNAfold (Markham and Zuker (2008) Methods Mol Biol. 453:3-31).

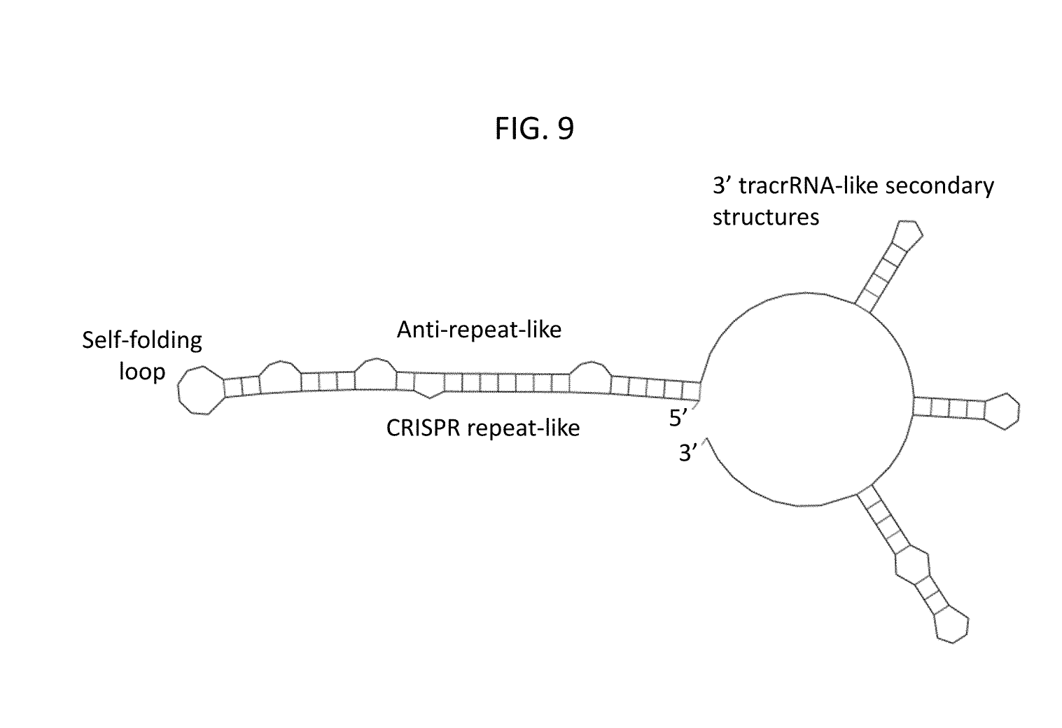

FIG. 9. Depiction of 5 prime secondary structure detected for putative tracrRNA (d) (SEQ ID NO: 102) from Lactobacillus apis (Lapis) CRISPR-Cas system when simulated to be transcribed in an anti-sense direction relative to the Lapis cas gene (SEQ ID NO: 113). RNA secondary structure was examined using UNAfold (Markham and Zuker (2008) Methods Mol Biol. 453:3-31).

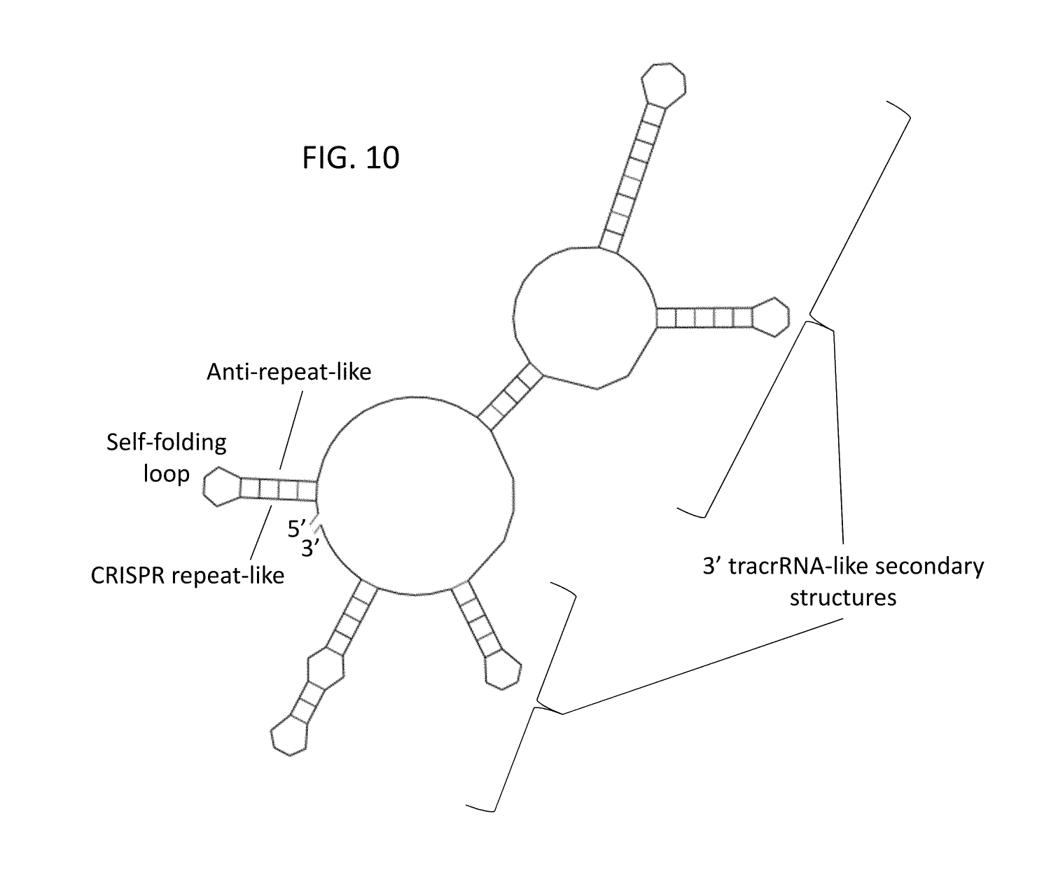

FIG. 10. Depiction of 5 prime secondary structure detected for putative tracrRNA (e) (SEQ ID NO: 103) from Lactobacillus apis (Lapis) CRISPR-Cas system when simulated to be transcribed in an anti-sense direction relative to the Lapis cas gene (SEQ ID NO: 114). RNA secondary structure was examined using UNAfold (Markham and Zuker (2008) Methods Mol Biol. 453:3-31).

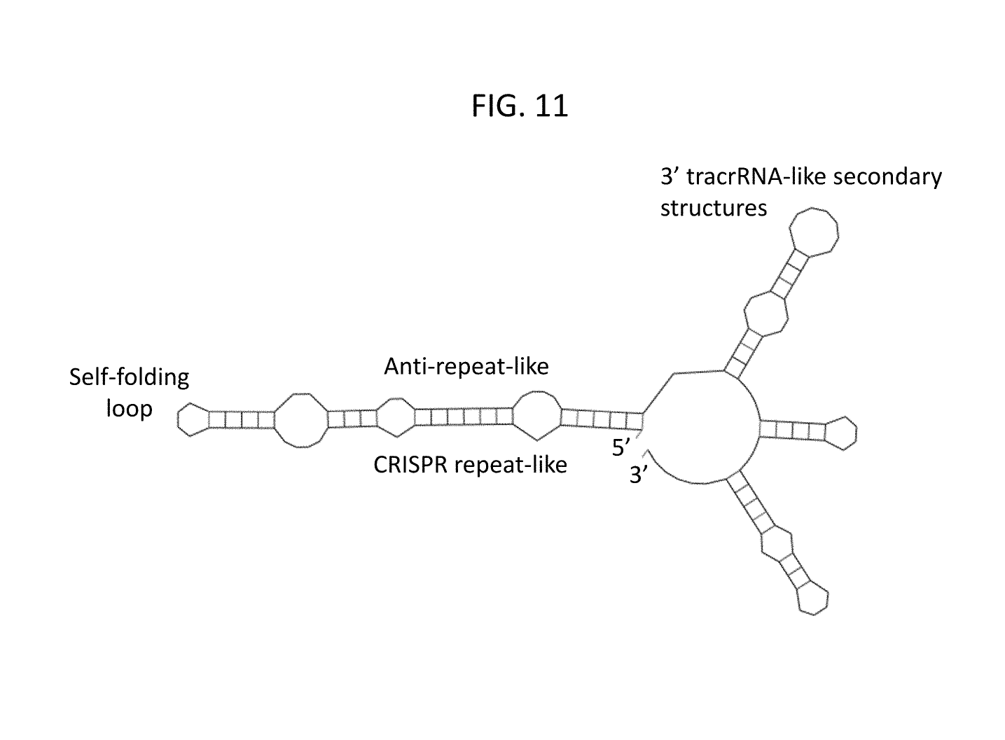

FIG. 11. Depiction of 5 prime secondary structure detected for putative tracrRNA (f) (SEQ ID NO: 104) from Lactobacillus apis (Lapis) CRISPR-Cas system when simulated to be transcribed in an anti-sense direction relative to the Lapis cas gene (SEQ ID NO: 115). RNA secondary structure was examined using UNAfold (Markham and Zuker (2008) Methods Mol Biol. 453:3-31).

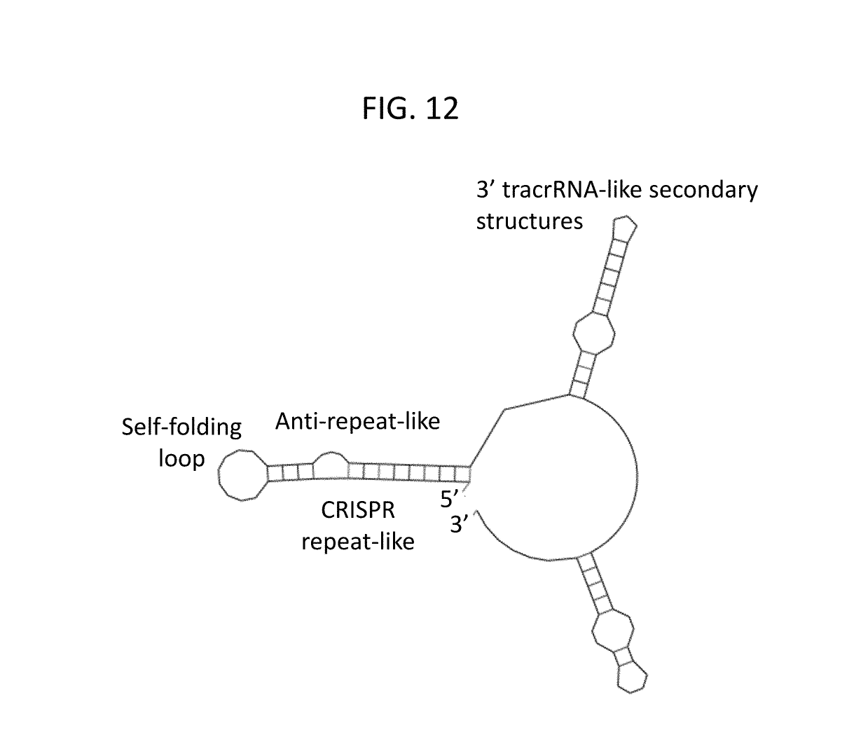

FIG. 12. Depiction of 5 prime secondary structure detected for putative tracrRNA (g) (SEQ ID NO: 105) from Lactobacillus apis (Lapis) CRISPR-Cas system when simulated to be transcribed in an anti-sense direction relative to the Lapis cas gene (SEQ ID NO: 116). RNA secondary structure was examined using UNAfold (Markham and Zuker (2008) Methods Mol Biol. 453:3-31).

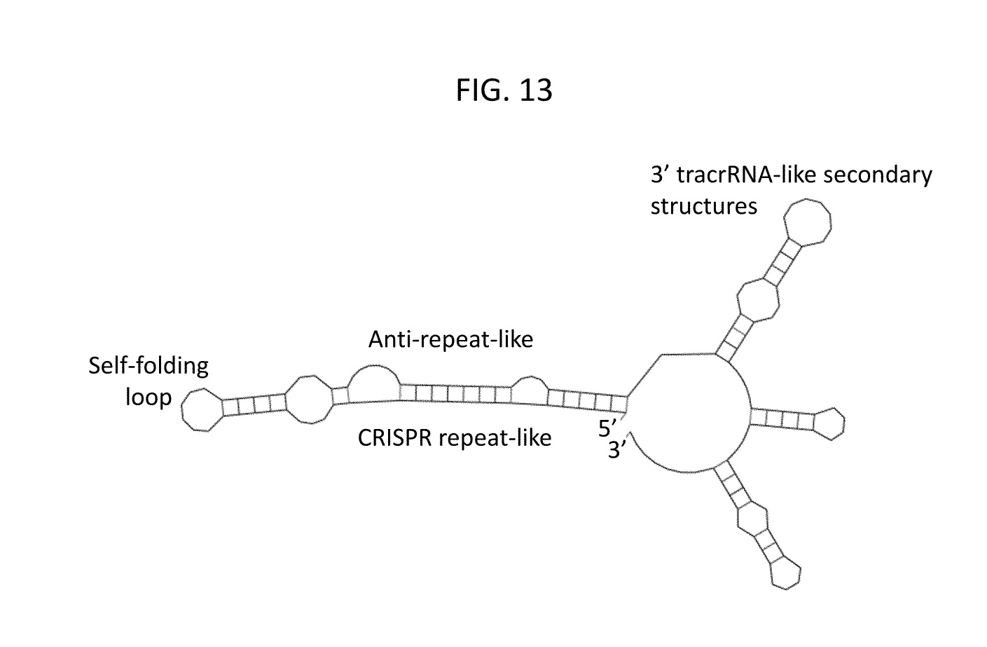

FIG. 13. Depiction of 5 prime secondary structure detected for putative tracrRNA (h) (SEQ ID NO: 106) from Lactobacillus apis (Lapis) CRISPR-Cas system when simulated to be transcribed in an anti-sense direction relative to the Lapis cas gene (SEQ ID NO: 117). RNA secondary structure was examined using UNAfold (Markham and Zuker (2008) Methods Mol Biol. 453:3-31).

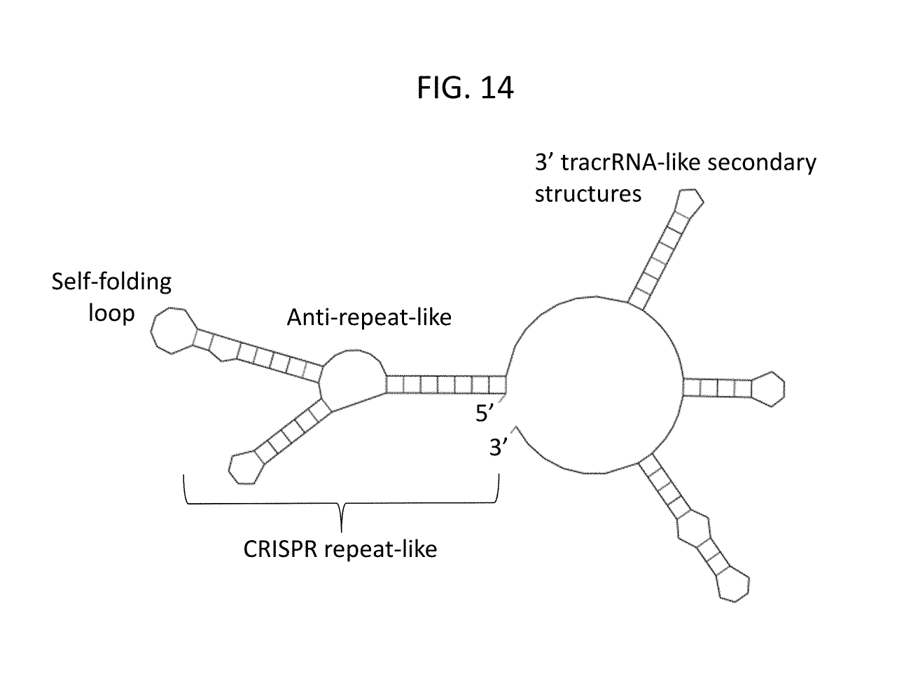

FIG. 14. Depiction of 5 prime secondary structure detected for putative tracrRNA (i) (SEQ ID NO: 107) from Lactobacillus apis (Lapis) CRISPR-Cas system when simulated to be transcribed in an anti-sense direction relative to the Lapis cas gene (SEQ ID NO: 118). RNA secondary structure was examined using UNAfold (Markham and Zuker (2008) Methods Mol Biol. 453:3-31).

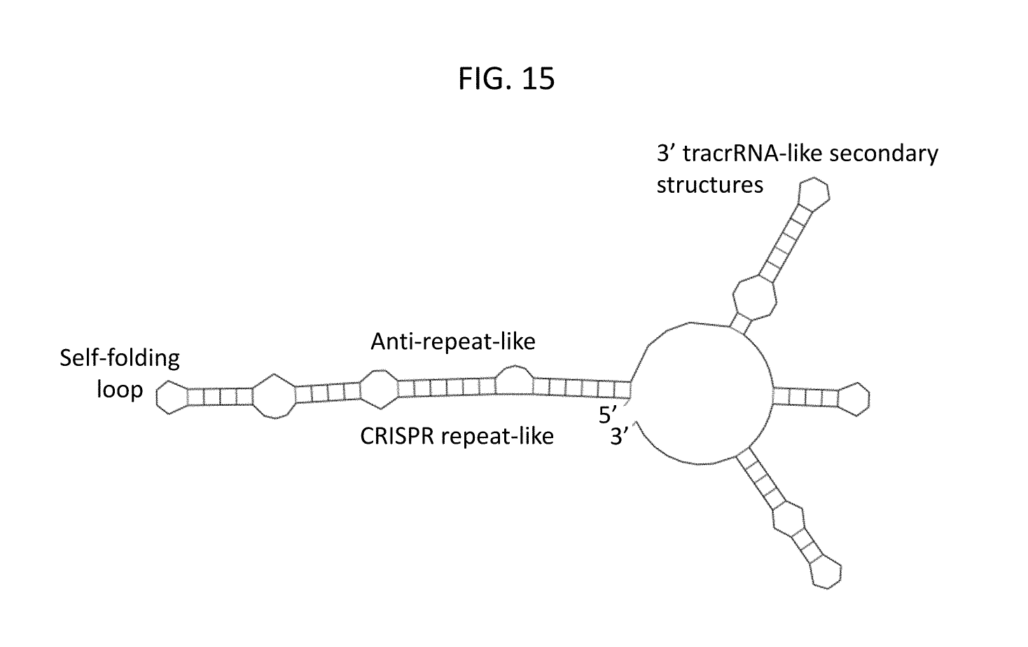

FIG. 15. Depiction of 5 prime secondary structure detected for putative tracrRNA (j) (SEQ ID NO: 108) from Lactobacillus apis (Lapis) CRISPR-Cas system when simulated to be transcribed in an anti-sense direction relative to the Lapis cas gene (SEQ ID NO: 119). RNA secondary structure was examined using UNAfold (Markham and Zuker (2008) Methods Mol Biol. 453:3-31).

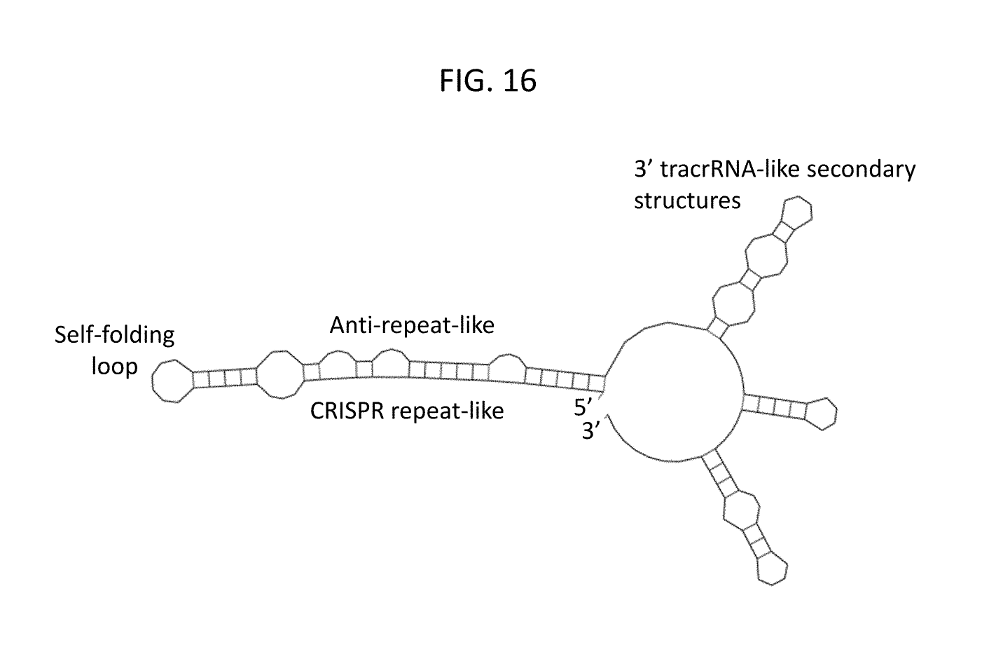

FIG. 16. Depiction of 5 prime secondary structure detected for putative tracrRNA (k) (SEQ ID NO: 109) from Lactobacillus apis (Lapis) CRISPR-Cas system when simulated to be transcribed in an anti-sense direction relative to the Lapis cas gene (SEQ ID NO: 120). RNA secondary structure was examined using UNAfold (Markham and Zuker (2008) Methods Mol Biol. 453:3-31).

### SEQUENCES

TABLE 1
Summary of Nucleic Acid and Amino Acid SEQ ID Numbers

| | Nucleic | Amino |
|---|---|---|
| | acid | Acid |
| | SEQ ID | SEQ ID |
| Description | NO: | NO: |
| Cas endonuclease protein from Lactobacillus apis | | 1 |
| (referred to as Lapis Cas) | | |
| Streptococcus pyogenes (Spy) M1 GAS Cas9 | | 2 |
| Streptococcus mutans UA159 Cas9 | | 3 |
| Streptococcus thermophilus LMD-9 Cas9 | | 4 |
| Streptococcus thermophilus LMD-9 Cas9 | | 5 |
| Lactobacillus rhamnosus GG Cas9 | | 6 |
| Veillonella atypica ACS-134-V-Col7a Cas9 | | 7 |
| Treponema denticola ATCC 35405 Cas9 | | 8 |
| Mycoplasma canis PG 14 Cas9 | | 9 |
| Enterococcus faecalis TX0012 Cas9 | | 10 |
| Mycoplasma gallisepticum str. F Cas9 | | 11 |
| Coriobacterium glomerans PW2 Cas9 | | 12 |
| Fusobacterium nucleatum ATCC 49256 Cas9 | | 13 |
| Finegoldia magna ATCC 29328 Cas9 | | 14 |
| Oenococcus kitaharae DSM 17330 Cas9 | | 15 |
| Peptoniphilus duerdenii ATCC BAA-1640 Cas9 | | 16 |
| Coprococcus catus GD-7 Cas9 | | 17 |
| Staphylococcus pseudintermedius ED99 Cas9 | | 18 |
| Bifidobacterium bifidum S17 Cas9 | | 19 |
| Streptococcus sanguinis SK49 Cas9 | | 20 |
| Eubacterium yurii ATCC 43715 Cas9 | | 21 |
| Acidaminococcus sp. D21 Cas9 | | 22 |
| Lactobacillus farciminis KCTC 3681 Cas9 | | 23 |
| Mycoplasma synoviae 53 Cas9 | | 24 |
| Eubacterium dolichum DSM 3991 Cas9 | | 25 |
| Eubacterium rectale ATCC 33656 Cas9 | | 26 |
| Staphylococcus lugdunensis M23590 Cas9 | | 27 |
| Filifactor alocis ATCC 35896 Cas9 | | 28 |
| Planococcus antarcticus DSM 14505> Cas9 | | 29 |
| Catenibacterium mitsuokai DSM 15897 Cas9 | | 30 |
| Solobacterium moorei F0204 Cas9 | | 31 |
| Fructobacillus fructosus KCTC 3544 Cas9 | | 32 |
| Mycoplasma ovipneumoniae SC01 Cas9 | | 33 |
| Mycoplasma mobile 163K Cas9 | | 34 |
| Francisella novicida U112 Cas9 | | 35 |
| Parasutterella excrementihominis YIT 11859 Cas9 | | 36 |
| Legionella pneumophila str. Paris Cas9 | | 37 |
| Wolinella succinogenes DSM 1740 Cas9 | | 38 |
| gamma proteobacterium HTCC5015 Cas9 | | 39 |
| Sutterella wadsworthensis 3 1 45B Cas9 | | 40 |
| Campylobacter jejuni NCTC 11168 Cas9 | | 41 |
| Neisseria meningitidis Z2491 Cas9 | | 42 |
| Pasteurella multocida str. Pm70 Cas9 | | 43 |
| Bacteroides sp. 20 3 Cas9 | | 44 |
| Bacteroides fragilis NCTC 9343 Cas9 | | 45 |
| Bifidobacterium longum DJO10A Cas9 | | 46 |
| Bacillus smithii 7 3 47FAA Cas9 | | 47 |
| Methylosinus trichosporium OB3b Cas9 | | 48 |
| Alicycliphilus denitrificans K601 Cas9 | | 49 |
| Prevotella timonensis CRIS 5C-B1 Cas9 | | 50 |
| Roseburia inulinivorans DSM 16841 Cas9 | | 51 |
| Prevotella sp. C561 Cas9 | | 52 |
| Dinoroseobacter shibae DFL 12 DSM 16493 Cas9 | | 53 |
| Flavobacterium branchiophilum FL-15 Cas9 | | 54 |
| Elusimicrobium minutum Pei191 Cas9 | | 55 |
| Ignavibacterium album JCM 16511 Cas9 | | 56 |
| Odoribacter laneus YIT 1206 Cas9 | | 57 |
| Caenispirillum salinarum | | 58 |
| Porphyromonas sp. oral taxon 279 str. F0450> | | 59 |
| Cas9 | | |
| Actinomyces sp. oral taxon 180 str. F031 Cas9 | | 60 |
| Sphaerochaeta globosa str. Buddy Cas9 | | 61 |
| Rhodospirillum rubrum ATCC 11170 Cas9 | | 62 |
| Azospirillum sp. B510 Cas9 | | 63 |
| Nitrobacter hamburgensis X14 Cas9 | | 64 |
| Ruminococcus albus 8 Cas9 | | 65 |
| Barnesiella intestinihominis YIT 11860 Cas9 | | 66 |
| Alicyclobacillus hesperidum URH17-3-68 Cas9 | | 67 |
| Acidothermus cellulolyticus 11B Cas9 | | 68 |
| Acidovorax ebreus TPSY Cas9 | | 69 |
| Lactobacillus coryniformis KCTC 3535 Cas9 | | 70 |
| Bergeyella zoohelcum ATCC 43767 Cas9 | | 71 |
| Alcanivorax pacificus W11-5 Cas9 | | 72 |
| Akkermansia muciniphila ATCC BAA-835 Cas9 | | 73 |
| Ilyobacter polytropus DSM 2926 Cas9 | | 74 |
| Bradyrhizobium sp. BTAi1 Cas9 | | 75 |
| Ralstonia syzygii R24 Cas9 | | 76 |
| Treponema sp. JC4 Cas9 | | 77 |
| Wolinella succinogenes DSM 1740 Cas9 | | 78 |
| Rhodovulum sp. PH10 Cas9 | | 79 |
| Aminomonas paucivorans DSM 12260 Cas9 | | 80 |
| Parvibaculum lavamentivorans DS-1 Cas9 | | 81 |
| Puniceispirillum marinum IMCC1322 Cas9 | | 82 |
| Helicobacter mustelae 12198 Cas9 | | 83 |
| Clostridium cellulolyticum H10 Cas9 | | 84 |
| uncultured delta proteobacterium HF0070 07E19 | | 85 |
| Cas9 | | |
| Nitratifractor salsuginis DSM 16511 Cas9 | | 86 |
| Actinomyces coleocanis DSM 15436 Cas9 | | 87 |
| Novel nuclease domain-1 of Lapis Cas (FIG. 1) | | 88 |
| Cas9 HNH domain consensus (FIG. 1) | | 89 |
| Novel nuclease domain-2, subdomain-1 of Lapis | | 90 |
| Cas (FIG. 2) | | |
| Cas9 Ruvc domain, subdomain-1 consensus | | 91 |
| (FIG. 2) | | |
| Novel nuclease domain-2, subdomain-2 of Lapis | | 92 |
| Cas (FIG. 3) | | |
| Cas9 Ruvc domain subdomain-2 consensus | | 93 |
| (FIG. 3) | | |
| Novel nuclease domain-2, subdomain-3 of Lapis | | 94 |
| Cas (FIG. 4) | | |
| Cas9 Ruvc domain, subdomain-3 consensus | | 95 |
| (FIG. 4) | | |
| CRISPR-Cas locus of Lactobacillus apis | 96 | |
| comprising the Lapis cas gene | | |
| Cas gene ORF from Lactobacillus apis (referred to | 97 | |
| as Lapis cas) | | |
| CRISPR repeat consensus from the Lactobacillus | 98 | |
| apis CRISPR-Cas system | | |
| Putative tracrRNA encoding region | 99-109 | |
| putative tracrRNA region a, b, c, d, e, f, g, h, l, j, k | 110-120 | |
| antisense transcriptional simulation | | |
| Randomized PAM library targeting sequence T1 | 121 | |
| Lapis putative tracrRNA e with T7 initiation | 122 | |
| sequence and T1 targeting sequence | | |
| Lapis putative tracrRNA h withT7 initiation | 123 | |
| sequence and T1 targeting sequence | | |
| Lapis putative tracrRNA i withT7 initiation | 124 | |
| sequence and T1 targeting sequence | | |
| T7 transcribed Lapis putative tracrRNA e with T7 | 125 | |
| initiation sequence and T1 targeting sequence | | |
| T7 transcribed Lapis putative tracrRNA h withT7 | 126 | |
| initiation sequence and T1 targeting sequence | | |
| T7 transcribed Lapis putative tracrRNA i withT7 | 127 | |
| initiation sequence and T1 targeting sequence | | |
| chimeric engineered guide RNAs derived from | 128-138 | |
| putative tracrRNAs described herein | | |

### DETAILED DESCRIPTION

Compositions are provided for novel Cas systems and elements comprising such systems, including, but not limiting to, novel guide polynucleotide/Cas endonucleases complexes, single guide polynucleotides, guide RNA elements, and Cas endonucleases. The present disclosure further includes compositions and methods for genome modification of a target sequence in the genome of a cell, for gene editing, and for inserting a polynucleotide of interest into the genome of a cell.

The term “cas gene” herein refers to one or more genes that are generally coupled, associated or close to, or in the vicinity of flanking CRISPR loci. The terms “Cas gene”, “CRISPR-associated (Cas) gene” and “Clustered Regularly Interspaced Short Palindromic Repeats-associated gene” are used interchangeably herein.

CRISPR (clustered regularly interspaced short palindromic repeats) loci refers to certain genetic loci encoding components of DNA cleavage systems, for example, used by bacterial and archaeal cells to destroy foreign DNA (Horvath and Barrangou, 2010, Science 327:167-170; WO2007/025097, published Mar. 1, 2007). A CRISPR locus can consist of a CRISPR array, comprising short direct repeats (CRISPR repeats) separated by short variable DNA sequences (called ‘spacers’), which can be flanked by diverse Cas (CRISPR-associated) genes. The number of CRISPR-associated genes at a given CRISPR locus can vary between species. Multiple CRISPR/Cas systems have been described including Class 1 systems, with multisubunit effector complexes (comprising type I, type III and type IV subtypes), and Class 2 systems, with single protein effectors (comprising type II and type V subtypes, such as but not limiting to Cas9, Cpf1, C2c1, C2c2, C2c3). Class 1 systems (Makarova et al. 2015, Nature Reviews; Microbiology Vol. 13:1-15; Zetsche et al., 2015, Cell 163, 1-13; Shmakov et al., 2015, Molecular_Cell 60, 1-13; Haft et al., 2005, Computational Biology, PLoS Comput Biol 1(6): e60. doi:10.1371/journal.pcbi. 0010060 and WO 2013/176772 A1 published on Nov. 23, 2013 incorporated by reference herein). The type II CRISPR/Cas system from bacteria employs a crRNA (CRISPR RNA) and tracrRNA (trans-encoding CRISPR RNA) to guide the Cas endonuclease to its DNA target. The crRNA contains a spacer region complementary to one strand of the double strand DNA target and a region that base pairs with the tracrRNA (trans-encoding CRISPR RNA) forming a RNA duplex that directs the Cas endonuclease to cleave the DNA target. Spacers are acquired through a not fully understood process involving Cas1 and Cas2 proteins. All type II CRISPR/Cas loci contain cas1 and cas2 genes in addition to the cas9 gene (Chylinski et al., 2013, RNA Biology 10:726-737; Makarova et al. 2015, Nature Reviews Microbiology Vol. 13:1-15). Type II CRISR-Cas loci can encode a tracrRNA, which is partially complementary to the repeats within the respective CRISPR array, and can comprise other proteins such as Csn1 and Csn2. The presence of cas9 in the vicinity of Cas 1 and cas2 genes is the hallmark of type II loci (Makarova et al. 2015, Nature Reviews Microbiology Vol. 13:1-15).

The term “Cas protein” refers to a protein encoded by a Cas (CRISPR-associated) gene. A Cas protein includes a Cas9 protein, a Cpf1 protein, a C2c1 protein, a C2c2 protein, a C2c3 protein, Cas3, Cas3-HD, Cas 5, Cas7, Cas8, Cas10, or combinations or complexes of these (Makarova et al. 2015, Nature Reviews Microbiology Vol. 13:1-15, Shmakov et al. 2017. Nature Reviews 15:169-182). A Cas protein includes Cas endonucleases. Cas endonucleases, when in complex with a suitable polynucleotide component, are capable of recognizing, binding to, and optionally nicking, cleaving, or covalently attaching to all or part of a specific DNA target sequence. A Cas endonuclease described herein comprises one or more nuclease domains.

“Cas9” (formerly referred to as Cas5, Csn1, or Csx12) refers to a Cas endonuclease that forms a complex with a crRNA and a tracrRNA, or with a single guide polynucleotide, for specifically recognizing and cleaving all or part of a DNA target sequence. A Cas9 protein comprises a RuvC nuclease domain and an HNH (H—N—H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al., 2013, Cell 157:1262-1278). Cas9 endonucleases are typically derived from a type II CRISPR system, which includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA (Makarova et al. 2015, Nature Reviews Microbiology Vol. 13:1-15).

As described herein, a CRISPR locus (SEQ ID NO:96) comprising a previously unidentified Cas endonuclease nucleotide sequence (referred to as Lapis cas gene; SEQ ID NO: 97), encoding a Cas endonuclease (referred to as Lapis Cas endonuclease, SEQ ID NO: 1) containing previously undefined endonuclease domains was identified from Lactobacillus apis (referred to herein as Lapis). The Lapis Cas endonuclease described herein represents a novel Cas endonuclease lacking the signature residues of a Cas9 endonuclease HNH domain as well as lacking signature residues of a Cas9 endonuclease RuvC domain (FIGS. 1-4, Example 1). The novel Cas endonuclease described herein (Lapis Cas endonuclease) comprises two previously unidentified nuclease domains, wherein the first nuclease domain is a nuclease domain of SEQ ID NO: 88, and the second nuclease domain is a nuclease domain comprising three subdomains of SEQ ID NO: 90, SEQ ID NO: 92 and SEQ ID NO: 94, respectively. The Lapis Cas endonuclease can form a complex with a guide polynucleotide, in which the ability to recognize, bind to, and optionally nick or cleave (introduce a single or double-strand break in) a target site is retained.

A “functional fragment”, “fragment that is functionally equivalent” and “functionally equivalent fragment” of a Lapis Cas nuclease domain or a Lapis cas nuclease subdomain are used interchangeably herein, and refer to a portion or subsequence of the nuclease domain or subdomain of the present disclosure in which the ability to covalently attach to, recognize, bind to, and optionally nick or cleave (introduce a single or double-strand break in) the target site is retained.

Functional fragments of a Lapis Cas endonuclease include fragments comprising 50-100, 100-200, 100-300, 100-400, 100-500, 100-600, 100-700, 100-800, 100-900, 100-1000, 200-300, 200-400, 200-500, 200-600, 200-700, 200-800, 200-900, 200-1000, 300-400, 300-500, 300-600, 300-700, 300-800, 300-900, 300-1000, 400-500, 400-600, 400-700, 400-800, 400-900, 400-1000, 500-600, 500-700, 500-800, 500-900, 500-1000, 600-700, 600-800, 600-900, 600-1000, 700-800, 700-900, 700-1000, 800-900, 800-1000, 900-1000, 1000-1100, 1100-1200 or 1200-1300 amino acids of a reference Lapis Cas protein, such as the reference Lapis Cas endonuclease of the present disclosure of SEQ ID NO: 1.

A variant of the Lapis Cas protein (SEQ ID NO: 1) and corresponding Lapis Cas gene (SEQ ID NO: 97) described herein may be used, but should have specific binding activity, and optionally endonucleolytic activity, towards DNA when associated with an RNA component herein. Such a variant Lapis Cas proteins may comprise an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of the reference Cas endonuclease of SEQ ID NO: 1. A variant Lapis Cas gene may comprise a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to the Lapis Cas endonuclease nucleotide sequence of SEQ ID NO: 97.

A variant of the Lapis Cas endonuclease described herein can comprise at least one of two domains, or at least both domains, wherein the first nuclease domain is a nuclease domain of SEQ ID NO: 88 or a functional fragment of SEQ ID NO: 88, and the second nuclease domain is a nuclease domain comprising at least one nuclease subdomain selected from the group consisting of SEQ ID NO: 90, SEQ ID NO: 92 and SEQ ID NO: 94, or a functional fragment of the second domain. The variant Lapis Cas endonuclease of the present disclosure can form a complex with a guide polynucleotide, in which the ability to covalently attach to, recognize, bind to, and optionally nick or cleave (introduce a single or double-strand break in) the target site is retained.

Fragments and variants can be obtained via methods such as site-directed mutagenesis and synthetic construction. Methods for measuring endonuclease activity are well known in the art such as, but not limiting to, PCT/US13/39011, filed May 1, 2013, PCT/US16/32073 filed May 12, 2016, PCT/US16/32028 filed May 12, 2016, incorporated by reference herein).

Methods for determining if fragments and/or variants of a Lapis Cas endonuclease of the present disclosure are functional include methods that measure the endonuclease activity of the fragment or variant when in complex with a suitable polynucleotide. Methods that measure endonuclease activity are well known in the art such as, but not limiting to, PCT/US13/39011, filed May 1, 2013, PCT/US16/32073 filed May 12, 2016, PCT/US16/32028 filed May 12, 2016, incorporated by reference herein). Methods for measuring Lapis Cas endonuclease activity include methods that measure the mutation frequency at a target site after a double strand break has occurred.

Methods for measuring Lapis Cas endonuclease activity include methods that measure the mutation frequency at a target site after a double strand break has occurred. Methods for measuring if a functional fragment or functional variant of a Lapis Cas endonuclease of the present disclosure can make a double strand break include the following method: briefly, appropriate CRISPR-Lapis Cas maize genomic DNA target sites can be selected, a guide RNA transcriptional cassette (recombinant DNA that expresses a guide RNA) and a DNA recombinant construct expressing the Lapis Cas endonuclease of the present disclosure (or a functional fragment of the Lapis Cas endonuclease of the present disclosure, or a functional variant of the Lapis Cas endonuclease variant of the present disclosure endonuclease can be constructed and can be co-delivered by biolistic transformation into Hi-Type II 10-day-old immature maize embryos (IMEs) in the presence of BBM and WUS2 genes as described in Svitashev et al. (2015). A visual marker DNA expression cassette encoding a yellow fluorescent protein can also be co-delivered with the guide RNA transcriptional cassette and the Lapis Cas endonuclease expression cassette (recombinant DNA construct) to aid in the selection of evenly transformed IMEs. After 2 days, the 20-30 most evenly transformed IMEs can be harvested based on their fluorescence. Total genomic DNA is extracted and the DNA region surrounding the intended target site is PCR amplified with Phusion® HighFidelity PCR Master Mix (New England Biolabs, M0531L) adding on the sequences necessary for amplicon-specific barcodes and Illumnia sequencing and deep sequenced. The resulting reads are then examined for the presence of mutations at the expected site of cleavage by comparison to control experiments where the guide RNA transcriptional cassette was omitted from the transformation. If mutations are observed at the intended target sites when using a fragment or variant of the Lapis Cas endonuclease of the present disclosure, in complex with a suitable guide polynucleotide, the fragments or variants are functional.

Methods for measuring if a functional fragment of functional variant of a Lapis Cas endonuclease of the present disclosure can make a single strand break (also referred to as a nick; hence acts as a nickase) in the double stranded DNA target site include the following method: The cellular repair of chromosomal single-strand breaks (SSBs) in a double-stranded DNA target may be typically repaired seamlessly in plant cells such as maize. Therefore, to examine a functional Lapis Cas fragment or functional variant of a Lapis Cas for nicking activity, two chromosomal DNA target sites in close proximity (0-200 bp), each targeting a different strand (sense and anti-sense DNA strands) of the double-stranded DNA, can be targeted. If SSB activity is present, the SSB activity from both target sites will result in a DNA double-strand break (DSB) that will result in the production of insertion or deletion (indel) mutagenesis in maize cells. This outcome can then be used to detect and monitor the activity of the Lapis Cas nickase similar to that described in Karvelis et al. (2015). Briefly, appropriate CRISPR-Lapis Cas maize genomic DNA target sites are selected, guide RNA transcription cassettes and functional fragment Lapis Cas nicking expression cassettes are constructed and co-delivered by biolistic transformation into Hi-Type II 10-day-old immature maize embryos (IMEs) in the presence of BBM and WUS2 genes as described in Svitashev et al. (2015). Since particle gun transformation can be highly variable, a visual marker DNA expression cassette encoding a yellow fluorescent protein can also be co-delivered to aid in the selection of evenly transformed IMEs [immature maize embryos]. After 2 days, the 20-30 most evenly transformed IMEs are harvested based on their fluorescence, total genomic DNA extracted, the region surrounding the intended target site PCR amplified with Phusion® HighFidelity PCR Master Mix (New England Biolabs, M0531 L) adding on the sequences necessary for amplicon-specific barcodes and Illumnia sequencing and deep sequenced. The resulting reads are then examined for the presence of mutations at the expected site of cleavage by comparison to control experiments where the small RNA transcriptional cassette was omitted from the transformation.

Methods for measuring if a functional fragment of functional variant of a Lapis Cas endonuclease of the present disclosure can bind to the intended DNA target site include the following method: The binding of a maize chromosomal DNA target site does not result in either a single-stranded break (SSB) or a double-stranded break (DSB) in the double-stranded DNA target site. Therefore, to examine a functional Lapis Cas fragment for binding activity in maize cells, another nuclease domain (e.g. FokI) may be attached to the functional Lapis Cas fragment with binding activity. If binding activity is present, the added nuclease domain may be used to produce a DSB that will result in the production of insertion or deletion (indel) mutagenesis in maize cells. This outcome may then be used to detect and monitor the binding activity of a Lapis Cas similar to that described in Karvelis et al. (2015). Briefly, appropriate CRISPR-Lapis Cas maize genomic DNA target sites can be selected, guide RNA transcription cassettes and functional fragment Lapis Cas binding and nuclease attached expression cassettes can be constructed and co-delivered by biolistic transformation into Hi-Type II 10-day-old immature maize embryos (IMEs) in the presence of BBM and WUS2 genes as described in Svitashev et al. (2015). A visual marker DNA expression cassette encoding a yellow fluorescent protein can also be co-delivered to aid in the selection of evenly transformed IMEs [immature maize embryos]. After 2 days, the 20-30 most evenly transformed IMEs can be harvested based on their fluorescence, total genomic DNA extracted, the region surrounding the intended target site PCR amplified with Phusion® HighFidelity PCR Master Mix (New England Biolabs, M0531 L) adding on the sequences necessary for amplicon-specific barcodes and Illumnia sequencing and deep sequenced. The resulting reads can then be examined for the presence of mutations at the expected site of cleavage by comparison to control experiments where the small RNA transcriptional cassette was omitted from the transformation.

Alternatively, the binding activity of maize chromosomal DNA target sites can be monitored by the transcriptional induction or repression of a gene. This can be accomplished by attaching a transcriptional activation or repression domain to the functional Lapis Cas binding fragment and targeting it to the promoter region of a gene and binding monitored through an increase in accumulation of the gene transcript or protein. The gene targeted for either activation or repression can be any naturally occurring maize gene or engineered gene (e.g. a gene encoded a red fluorescent protein) introduced into the maize genome by methods known in the art (e.g. particle gun or agrobacterium transformation).

Cas endonucleases, including the Lapis Cas endonuclease described herein, can be used for targeted genome editing (via simplex and multiplex double-strand breaks and nicks) and targeted genome regulation (via tethering of epigenetic effector domains to either the Cas protein or sgRNA. A Cas endonuclease can also be engineered to function as an RNA-guided recombinase, and via RNA tethers could serve as a scaffold for the assembly of multiprotein and nucleic acid complexes (Mali et al., 2013, Nature Methods Vol. 10: 957-963).

The term “plant-optimized Lapis Cas endonuclease” herein refers to a Lapis Cas protein encoded by a nucleotide sequence that has been optimized for expression in a plant cell or plant.

The Lapis Cas protein, or functional fragment thereof, for use in the disclosed methods, can be isolated from a recombinant source where the genetically modified host cell (e.g. an insect cell or a yeast cell or human-derived cell line) is modified to express the nucleic acid sequence encoding the Cpf1 protein. Alternatively, the Lapis Cas protein can be produced using cell free protein expression systems or be synthetically produced.

A “plant-optimized nucleotide sequence encoding a Lapis Cas endonuclease”, “plant-optimized construct encoding a Lapis Cas endonuclease” and a “plant-optimized polynucleotide encoding a Lapis Cas” are used interchangeably herein and refer to a nucleotide sequence encoding an Lapis Cas protein, or a variant or functional fragment thereof, that has been optimized for expression in a plant cell or plant. A plant comprising a plant-optimized Lapis Cas endonuclease includes a plant comprising the nucleotide sequence encoding for the Lapis Cas sequence and/or a plant comprising the Lapis Cas endonuclease protein. In one aspect, the plant-optimized Lapis Cas endonuclease nucleotide sequence is a maize-optimized, rice-optimized, wheat-optimized or soybean-optimized Lapis Cas endonuclease.

The Cas endonuclease, including the Lapis Cas endonuclease described herein, can comprise a modified form of the Cas polypeptide. The modified form of the Cas polypeptide can include an amino acid change (e.g., deletion, insertion, or substitution) that reduces the naturally-occurring nuclease activity of the Cas protein. For example, in some instances, the modified form of the Cas protein has less than 50%, less than 40%, less than 30%, less than 20%, less than 10%, less than 5%, or less than 1% of the nuclease activity of the corresponding wild-type Cas polypeptide (US patent application US20140068797 A1, published on Mar. 6, 2014). In some cases, the modified form of the Cas polypeptide has no substantial nuclease activity and is referred to as catalytically “inactivated Cas” or “deactivated Cas (dCas).” An inactivated Cas/deactivated Cas includes a deactivated Lapis Cas endonuclease (Lapis dCas).

A catalytically inactive Lapis Cas can be fused to a heterologous sequence as described herein.

The terms “functional fragment”, “fragment that is functionally equivalent” and “functionally equivalent fragment” of a Cas endonuclease are used interchangeably herein, and refer to a portion or subsequence of the Cas endonuclease sequence, including the Lapis Cas endonuclease of the present disclosure in which the ability to covalently attach to, recognize, bind to, and optionally nick or cleave (introduce a single or double-strand break in) the target site is retained.

The terms “functional variant”, “Variant that is functionally equivalent” and “functionally equivalent variant” of a Cas endonuclease, including the Lapis Cas endonuclease of the present disclosure, are used interchangeably herein, and refer to a variant of the Cas endonuclease of the present disclosure in which the ability to recognize, bind to, and optionally nick or cleave (introduce a single or double-strand break in) the target site is retained. Fragments and variants can be obtained via methods such as site-directed mutagenesis and synthetic construction.

A Lapis Cas protein, such as the Lapis Cas endonuclease described herein, can comprise at least one heterologous nuclear localization sequence (NLS). A heterologous NLS amino acid sequence herein may be of sufficient strength to drive accumulation of the Lapis Cas protein described herein, in a detectable amount in the nucleus of a eukaryotic cell. An NLS may comprise one (monopartite) or more (e.g., bipartite) short sequences (e.g., 2 to 20 residues) of basic, positively charged residues (e.g., lysine and/or arginine), and can be located anywhere in a Cas amino acid sequence but such that it is exposed on the protein surface. An NLS may be operably linked to the N-terminus or C-terminus of a Cas protein herein, for example. Two or more NLS sequences can be linked to a Cas protein, for example, such as on both the N- and C-termini of a Cas protein. The Cas endonuclease gene can be operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region. Non-limiting examples of suitable NLS sequences herein include the Simian virus 40 (SV40) NLS, a Penetratin, a bipartite NLS or the RNP A1 NLS (M9 region), or endogenous NLSs as disclosed in U.S. Pat. Nos. 6,660,830 and 7,309,576, which are both incorporated by reference herein.

Conventional NLSs are short peptide sequences that facilitate nuclear localization of the proteins containing them (see for example, Human a1 T-ag, CBP80, DNA helicase Q1, BRCA1, Mitosin, Myc, NF-kB p50, NF-kB p65, H1V1422, HIV1423, Human a2 T-ag, NF-kB p50, DNA helicase Q1, LEF-1, EBNA1, HIV-1 IN, HIV-1 MA, H1V1422, HIV1423, RCP 4.1R, Human a3 T-ag, DNA helicase 01, tTS, Human a4 T-ag, Mouse a1 LEF-1, Mouse a2, T-agáCK2 site, Impa-P1) T-ag, N1N2, RB, Dorsal áPK\check{}A site, CBP80, DNA helicase Q1, LEF-1, Mouse a2, T-agáCK2, Impa-P1) T-ag, N1N2, RB, Dorsal áPK{hacek over 0}A, CBP80, DNA helicase Q1, LEF-1, Xenopus a1 T-ag, Nucleoplasmin, Yeast a1 T-ag, (SRP1, Kap60), T-agáCK2, N1N2, HIV-1 IN, Plant a1 T-ag, T-agáCK2, Opaque-2, R Protein (Maize), N1N2, RAG-1, RCP, RB, STAT, CBP80, LEF, EBNA, IN, tTG, tissue ICP, described inTable 1 of U.S. Pat. No. 7,309,576, incorporated by reference herein, and Jans et al., 2000, BioEssays 22:532-544). Any NLS may be employed in the methods described herein. Nucleotide sequences encoding a selected NLS may be derived from the amino acid sequence of the NLS and are synthesized and incorporated into the nucleotide sequence encoding the Cas endonuclease described herein by conventional methods.

A Cas protein herein such as a Lapis Cas protein can comprise a heterologous nuclear localization sequence (NLS) on either or both N- and C-terminus as well as a N-terminal or C-terminal tag such as but not limiting to a N-terminal 6×His Tag. N-terminal or C-terminal tags can be used for purification of the Cas endonuclease protein described herein.

A Cas protein, including the Lapis Cas endonuclease described herein, can be part of a fusion protein comprising one or more heterologous protein domains (e.g., 1, 2, 3, or more domains in addition to the Cas protein). Such a fusion protein may comprise any additional protein sequence, and optionally a linker sequence between any two domains, such as between Cas and a first heterologous domain. Examples of protein domains that may be fused to a Cas protein herein include, without limitation, epitope tags (e.g., histidine [His], V5, FLAG, influenza hemagglutinin [HA], myc, VSV-G, thioredoxin [Trx]), reporters (e.g., glutathione-5-transferase [GST], horseradish peroxidase [HRP], chloramphenicol acetyltransferase [CAT], beta-galactosidase, beta-glucuronidase [GUS], luciferase, green fluorescent protein [GFP], HcRed, DsRed, cyan fluorescent protein [CFP], yellow fluorescent protein [YFP], blue fluorescent protein [BFP]), and domains having one or more of the following activities: methylase activity, demethylase activity, transcription activation activity (e.g., VP16 or VP64), transcription repression activity, transcription release factor activity, histone modification activity, RNA cleavage activity and nucleic acid binding activity. A Cas protein can also be in fusion with a protein that binds DNA molecules or other molecules, such as maltose binding protein (MBP), S-tag, Lex A DNA binding domain (DBD), GAL4A DNA binding domain, and herpes simplex virus (HSV) VP16.

A catalytically inactive Cas, including a catalytically inactive Lapis Cas endonuclease, can be fused to a heterologous sequence (US patent application US20140068797 A1, published on Mar. 6, 2014). Suitable fusion partners include, but are not limited to, a polypeptide that provides an activity that indirectly increases transcription by acting directly on the target DNA or on a polypeptide (e.g., a histone or other DNA-binding protein) associated with the target DNA. Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.). A catalytically inactive Cas9 can also be fused to a FokI nuclease to generate double-strand breaks (Guilinger et al. Nature biotechnology, volume 32, number 6, June 2014).

As used herein, the term “guide polynucleotide”, relates to a polynucleotide sequence that can form a complex with a Cas endonuclease, including the Lapis Cas endonuclease described herein, and enables the Cas endonuclease to recognize, bind to, and optionally nick, cleave, or covalently attach to a DNA target site. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA” (See also U.S. Patent Application US20150082478, published on Mar. 19, 2015 and US20150059010, published on Feb. 26, 2015, both are incorporated by reference herein).

The guide polynucleotide comprises a first nucleotide sequence domain that is recognized by a Cas endonuclease (such as a Lapis Cas endonuclease), referred to as Cas endonuclease recognition domain (CER domain; Lapis Cas recognition domain for Lapis Cas endonucleases) and a Variable Targeting domain or VT domain that can hybridize to a nucleotide sequence in a target DNA. By “domain” it is meant a contiguous stretch of nucleotides that can be RNA, DNA, or a RNA-DNA-combination sequence. The VT domain and/or the CER domain of a guide polynucleotide can comprise a RNA sequence, a DNA sequence, or a RNA-DNA-combination sequence. The guide polynucleotide may be referred to as “guide RNA” (when composed of a contiguous stretch of RNA nucleotides) or “guide DNA” (when composed of a contiguous stretch of DNA nucleotides) or “guide RNA-DNA” (when composed of a combination of RNA and DNA nucleotides). In one aspect, the guide polynucleotide can form a complex with a Lapis Cas endonuclease, wherein said guide polynucleotide/Lapis Cas endonuclease complex (also referred to as a guide polynucleotide/Lapis Cas endonuclease system) can direct the Lapis Cas endonuclease to a genomic target site, enabling the Lapis Cas endonuclease to recognize, bind to, and optionally nick or cleave (introduce a single or double-strand break) the target site. The guide polynucleotide includes a chimeric engineered guide RNA. The term “chimeric engineered guide RNA” relates to a polynucleotide sequence that is engineered to comprise regions that are not found together in nature (i.e., they are heterologous with each other) and can form a complex with a Cas endonuclease, including the Lapis Cas endonuclease described herein, and enables the Cas endonuclease to recognize, bind to, and optionally nick, cleave, or covalently attach to a DNA target site. For example, a chimeric engineered guide RNA can be engineered to comprise a first RNA nucleotide sequence domain (referred to as Variable Targeting domain or VT domain) that can hybridize to a nucleotide sequence in a target DNA, linked to a second RNA nucleotide sequence that can be recognized by the Lapis Cas endonuclease (such as the putative tracrRNA like sequences described herein), such that the first and second nucleotide sequence are not found linked together in nature.

The guide polynucleotide, capable of directing the Lapis Cas endonuclease to a target sequence, contains a nucleotide sequence with homology to a DNA target sequence (also referred to as a variable targeting domain) 5 prime to any one of the putative tracrRNAs described herein (Example 2). Examples of such chimeric engineered guide RNAs are shown in SEQ ID NOs: 128-138 where N may be any nucleotide and wherein the 5 prime sequence of Ns can vary from 12, 13, 14, 15, 16, 17, 18, 19, 20, 21, 22, 23, 24, 25, 26, 27, 28, 29 or 30 in length. As shown in FIGS. 6-16, the putative tracrRNAs contained a CRISPR repeat-like sequence, a loop sequence that promoted self-folding, an anti-repeat-like sequence with partial complementation to the repeat sequence, and a 3 prime region with tracrRNA hairpin-like secondary structures.

The term “variable targeting domain” or “VT domain” is used interchangeably herein and includes a nucleotide sequence that can hybridize (is complementary) to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The variable targeting domain replaces the spacer sequence normally found in the native Lapis CRISPR locus (SEQ ID NO: 96).

In some embodiments, the variable targeting domain comprises a contiguous stretch of 12 to 30, 12 to 29, 12 to 28, 12 to 27, 12 to 26, 12 to 25, 12 to 26, 12 to 25, 12 to 24, 12 to 23, 12 to 22, 12 to 21, 12 to 20, 12 to 19, 12 to 18, 12 to 17, 12 to 16, 12 to 15, 12 to 14, 12 to 13, 13 to 30, 13 to 29, 13 to 28, 13 to 27, 13 to 26, 13 to 25, 13 to 26, 13 to 25, 13 to 24, 13 to 23, 13 to 22, 13 to 21, 13 to 20, 13 to 19, 13 to 18, 13 to 17, 13 to 16, 13 to 15, 13 to 14, 14 to 30, 14 to 29, 14 to 28, 14 to 27, 14 to 26, 14 to 25, 14 to 26, 14 to 25, 14 to 24, 14 to 23, 14 to 22, 14 to 21, 14 to 20, 14 to 19, 14 to 18, 14 to 17, 14 to 16, 14 to 15, 15 to 30, 15 to 29, 15 to 28, 15 to 27, 15 to 26, 15 to 25, 15 to 26, 15 to 25, 15 to 24, 15 to 23, 15 to 22, 15 to 21, 15 to 20, 15 to 19, 15 to 18, 15 to 17, 15 to 16, 16 to 30, 16 to 29, 16 to 28, 16 to 27, 16 to 26, 16 to 25, 16 to 24, 16 to 23, 16 to 22, 16 to 21, 16 to 20, 16 to 19, 16 to 18, 16 to 17, 17 to 30, 17 to 29, 17 to 28, 17 to 27, 17 to 26, 17 to 25, 17 to 24, 17 to 23, 17 to 22, 17 to 21, 17 to 20, 17 to 19, 17 to 18, 18 to 30, 18 to 29, 18 to 28, 18 to 27, 18 to 26, 18 to 25, 18 to 24, 18 to 23, 18 to 22, 18 to 21, 18 to 20, 18 to 19, 19 to 30, 19 to 29, 19 to 28, 19 to 27, 19 to 26, 19 to 25, 19 to 24, 19 to 23, 19 to 22, 19 to 21, 19 to 20, 20 to 30, 20 to 29, 20 to 28, 20 to 27, 20 to 26, 20 to 25, 20 to 24, 20 to 23, 20 to 22, 20 to 21, 21 to 30, 21 to 29, 21 to 28, 21 to 27, 21 to 26, 21 to 25, 21 to 24, 21 to 23, 21 to 22, 22 to 30, 22 to 29, 22 to 28, 22 to 27, 22 to 26, 22 to 25, 22 to 24, 22 to 23, 23 to 30, 23 to 29, 23 to 28, 23 to 27, 23 to 26, 23 to 25, 23 to 24, 24 to 30, 24 to 29, 24 to 28, 24 to 27, 24 to 26, 24 to 25, 25 to 30, 25 to 29, 25 to 28, 25 to 27, 25 to 26, 26 to 30, 26 to 29, 26 to 28, 26 to 27, 27 to 30, 27 to 29, 27 to 28, 28 to 30, 28 to 29, or 29 to 30 nucleotides.

The variable targeting domain can be composed of a DNA sequence, a RNA sequence, a modified DNA sequence, a modified RNA sequence, a RNA-DNA combination sequence, or any combination thereof.

The terms “functional fragment”, “fragment that is functionally equivalent” and “functionally equivalent fragment” of a guide RNA or putative tracrRNA are used interchangeably herein, and refer to a portion or subsequence of the guide RNA or putative tracrRNA, respectively, of the present disclosure in which the ability to function as a guide RNA or putative tracrRNA, respectively, is retained.

The terms “functional variant”, “Variant that is functionally equivalent” and “functionally equivalent variant” of a guide RNA or putative tracrRNA (respectively) are used interchangeably herein, and refer to a variant of the guide RNA or putative tracrRNA, respectively, of the present disclosure in which the ability to function as a guide RNA or putative tracrRNA, respectively, is retained.

The guide polynucleotide can be produced by any method known in the art, including chemically synthesizing guide polynucleotides (such as but not limiting to Hendel et al. 2015, Nature Biotechnology 33, 985-989), in vitro generated guide polynucleotides, and/or self-splicing guide RNAs (such as but not limiting to Xie et al. 2015, PNAS 112:3570-3575).

A functional fragments of a guide RNA or guide polynucleotide of the present disclosure include a fragment of 20-40, 20-45, 20-50, 20-55, 20-60, 20-65, 20-70, 20-75, 20-80, 25-40, 25-45, 25-50, 25-55, 25-60, 25-65, 25-70, 25-75, 25-80, 30-40, 30-45, 30-50, 30-55, 30-60, 30-65, 30-70, 30-75, 30-80, 35-40, 35-45, 35-50, 35-55, 35-60, 35-65, 35-70, 35-75, 35-80, 40-45, 40-50, 40-55, 40-60, 40-65, 40-70, 40-75, 40-80, 45-50, 45-55, 45-60, 45-65, 45-70, 45-75, 45-80, 50-55, 50-60, 50-65, 50-70, 50-75, 50-80, 55-55, 55-60, 55-65, 55-70, 55-75, 55-80, 60-65, 60-70, 60-75, 60-80, 65-70, 65-75, 65-80, 70-75, 70-80 or 75-80 nucleotides of a reference guide RNA, such as the reference guide RNAs of SEQ ID NOs: 128-138.

A functional variant of a single guide RNA may comprise a nucleotide sequence that is at least about 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to reference single guide RNA, such as the reference single guide RNA of SEQ ID NOs: 128-138, described herein. In some embodiments, a functional variant of a single guide RNA comprises a nucleotide sequence having at least about 60%, at least about 65%, at least about 70%, at least about 75%, at least about 80%, at least about 85%, at least about 90%, at least about 95%, at least about 98%, at least about 99%, or 100% nucleotide sequence identity over a stretch of at least 8, 9, 10, 11, 12, 13, 14, 15, 16, 17, 18, 19, 20, 21, 22, 23, 24, 25, 26, 27, 28, 29, 30 contiguous nucleotides to any one of the nucleotide sequences set forth in SEQ ID NOs: 128-138.

Nucleotide sequence modification of the guide polynucleotide, can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-0-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

The terms “5′-cap” and “7-methylguanylate (m⁷G) cap” are used interchangeably herein. A 7-methylguanylate residue is located on the 5′ terminus of messenger RNA (mRNA) in eukaryotes. RNA polymerase II (Pol II) transcribes mRNA in eukaryotes. Messenger RNA capping occurs generally as follows: The most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5′-5′ triphosphate-linked guanine at the transcript terminus. Finally, the 7-nitrogen of this terminal guanine is methylated by a methyl transferase.

The terminology “not having a 5′-cap” herein is used to refer to RNA having, for example, a 5′-hydroxyl group instead of a 5′-cap. Such RNA can be referred to as “uncapped RNA”, for example. Uncapped RNA can better accumulate in the nucleus following transcription, since 5′-capped RNA is subject to nuclear export. One or more RNA components herein are uncapped.

As used herein, the terms “guide polynucleotide/Cas endonuclease complex”, “guide polynucleotide/Cas endonuclease system”, “guide polynucleotide/Cas complex”, “guide polynucleotide/Cas system” and “guided Cas system” “Polynucleotide-guided endonuclease”, “PGEN” are used interchangeably herein and refer to at least one guide polynucleotide (such as a chimeric engineered guide RNA described herein) and at least one Cas endonuclease that are capable of forming a complex, wherein said guide polynucleotide/Cas endonuclease complex can direct the Cas endonuclease to a DNA target site, enabling the Cas endonuclease to recognize, bind to, and optionally nick, cleave (introduce a single or double-strand break), or covalently attach to the DNA target site. A Cas endonuclease unwinds the DNA duplex at the target sequence and optionally cleaves at least one DNA strand, as mediated by recognition of the target sequence by a polynucleotide that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component. (See also U.S. Patent Application US20150082478, published on Mar. 19, 2015 and US20150059010, published on Feb. 26, 2015, both are incorporated by reference herein).

A guide polynucleotide/Cas endonuclease complex includes a guide RNA/Cas endonuclease complex comprising at least one chimeric engineered guide RNA and a Cas endonuclease, wherein said Cas endonuclease comprises a first nuclease domain of SEQ ID NO: 88 or a functional fragment of SEQ ID NO: 88, and a second nuclease domain comprising at least one nuclease subdomain selected from the group consisting of SEQ ID NO: 90, SEQ ID NO: 92 and SEQ ID NO: 94, or a functional fragment of said second domain, wherein said guide RNA/Cas endonuclease complex is capable of recognizing, binding to, and optionally nicking, cleaving, or covalently attaching to all or part of a target sequence.

A guide polynucleotide/Cas endonuclease complex includes a guide RNA/Cas endonuclease complex comprising a Cas endonuclease of SEQ ID NO: 1, or a functional fragment thereof, and at least one chimeric engineered guide RNA, wherein said guide RNA/Cas endonuclease complex is capable of recognizing, binding to, and optionally nicking, cleaving, or covalently attaching to all or part of a target sequence

A guide polynucleotide/Cas endonuclease complex includes a guide RNA/Cas endonuclease complex comprising at least one chimeric engineered guide RNA and a Cas endonuclease, wherein said Cas endonuclease is encoded by a eukaryotic codon optimized sequence of SEQ ID NO: 97, wherein said guide RNA/Cas endonuclease complex is capable of recognizing, binding to, and optionally nicking, cleaving, or covalently attaching to all or part of a target sequence.

A guide polynucleotide/Cas endonuclease complex, including a guide polynucleotide/Lapis Cas endonuclease complex described herein, can cleave one or both strands of a DNA target sequence.

A guide polynucleotide/Cas endonuclease complex that can cleave both strands of a DNA target sequence typically comprises a Cas protein that has all of its endonuclease domains in a functional state (e.g., wild type endonuclease domains or variants thereof retaining some or all activity in each endonuclease domain).

A guide polynucleotide/Cas endonuclease complex that can cleave one strand of a DNA target sequence can be characterized herein as having nickase activity (e.g., partial cleaving capability). A Cas nickase typically comprises one functional endonuclease domain that allows the Cas to cleave only one strand (i.e., make a nick) of a DNA target sequence. A pair of Cas nickases can be used to increase the specificity of DNA targeting. In general, this can be done by providing two Cas nickases that, by virtue of being associated with RNA components with different guide sequences, target and nick nearby DNA sequences on opposite strands in the region for desired targeting. Such nearby cleavage of each DNA strand creates a double-strand break (i.e., a DSB with single-stranded overhangs), which is then recognized as a substrate for non-homologous-end-joining, NHEJ (prone to imperfect repair leading to mutations) or homologous recombination, HR. Each nick in these embodiments can be at least about 5, 10, 15, 20, 30, 40, 50, 60, 70, 80, 90, or 100 (or any integer between 5 and 100) bases apart from each other, for example. One or two Cas nickase proteins herein can be used in a Cas nickase pair. A guide polynucleotide/Cas endonuclease complex in certain embodiments can bind to a DNA target site sequence, but does not cleave any strand at the target site sequence. Such a complex may comprise a Cas protein in which all of its nuclease domains are mutant, dysfunctional. A Cas protein that binds, but does not cleave, a target DNA sequence can be used to modulate gene expression, for example, in which case the Cas protein could be fused with a transcription factor (or portion thereof) (e.g., a repressor or activator, such as any of those disclosed herein).

The terms “guide RNA/Cas endonuclease complex”, “guide RNA/Cas endonuclease system”, “guide RNA/Cas complex”, “guide RNA/Cas system”, “gRNA/Cas complex”, “g RNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease, including the Lapis Cas endonuclease described herein, to a DNA target site, enabling the Cas endonuclease to covalently attach to, recognize, bind to, and optionally nick or cleave (introduce a single or double-strand break) the DNA target site.

The present disclosure further provides expression constructs for expressing in a prokaryotic or eukaryotic cell/organism a guide RNA/Cas system that is capable of binding to and creating a double-strand break in a target site. In one embodiment, the expression constructs of the disclosure comprise a promoter operably linked to a nucleotide sequence encoding a Lapis Cas gene (or plant or mammalian optimized Lapis Cas gene) and a promoter operably linked to a guide RNA of the present disclosure. The promoter is capable of driving expression of an operably linked nucleotide sequence in a prokaryotic or eukaryotic cell/organism.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a transgenic locus, or any other DNA molecule in the genome (including chromosomal, choloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

Methods for “modifying a target site” and “altering a target site” are used interchangeably herein and refer to methods for producing an altered target site.

The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas endonuclease.

Assays to measure the single or double-strand break of a target site by an endonuclease are known in the art and generally measure the overall activity and specificity of the agent on DNA substrates containing recognition sites.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

The guide polynucleotide/Cas systems described herein can be used for gene targeting.

The terms “gene targeting”, “targeting”, and “DNA targeting” are used interchangeably herein. DNA targeting herein may be the specific introduction of a knock-out, edit, or knock-in at a particular DNA sequence, such as in a chromosome or plasmid of a cell. In general, DNA targeting can be performed herein by cleaving one or both strands at a specific DNA sequence in a cell with a Cas protein associated with a suitable polynucleotide component. Once a single or double-strand break is induced in the DNA, the cell's DNA repair mechanism is activated to repair the break via nonhomologous end-joining (NHEJ) or Homology-Directed Repair (HDR) processes which can lead to modifications at the target site.

The terms “knock-out”, “gene knock-out” and “genetic knock-out” are used interchangeably herein. A knock-out represents a DNA sequence of a cell that has been rendered partially or completely inoperative by targeting with a Cas protein; such a DNA sequence prior to knock-out could have encoded an amino acid sequence, or could have had a regulatory function (e.g., promoter), for example.

As described herein, a guided Cas endonuclease can recognize, bind to a DNA target sequence and introduce a single strand (nick) or double-strand break. Once a single or double-strand break is induced in the DNA, the cell's DNA repair mechanism is activated to repair the break. Error-prone DNA repair mechanisms can produce mutations at double-strand break sites. The most common repair mechanism to bring the broken ends together is the nonhomologous end-joining (NHEJ) pathway (Bleuyard et al., (2006) DNA Repair 5:1-12). The structural integrity of chromosomes is typically preserved by the repair, but deletions, insertions, or other rearrangements (such as chromosomal translocations) are possible (Siebert and Puchta, 2002, Plant Cell 14:1121-31; Pacher et al., 2007, Genetics 175:21-9).

A knock-out may be produced by an indel (insertion or deletion of nucleotide bases in a target DNA sequence through NHEJ), or by specific removal of sequence that reduces or completely destroys the function of sequence at or near the targeting site.

In one embodiment, the disclosure describes a method for modifying a target site in the genome of a cell, the method comprising introducing into said cell at least one chimeric engineered guide RNA, and a Cas endonuclease comprising a first nuclease domain of SEQ ID NO: 88, and a second nuclease domain comprising at least one nuclease subdomain selected from the group consisting of SEQ ID NO: 90, SEQ ID NO: 92 and SEQ ID NO: 94, wherein said chimeric engineered guide RNA and Cas endonuclease can form a complex that is capable of recognizing, binding to, and optionally nicking, cleaving, or covalently attaching to all or part of said target site.

The guide polynucleotide/Cas endonuclease system can be used in combination with at least one polynucleotide modification template to allow for editing (modification) of a genomic nucleotide sequence of interest. (See also U.S. Patent Application US20150082478, published on Mar. 19, 2015 and WO2015/026886, published on Feb. 26, 2015, both are incorporated by reference herein.)

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

In one embodiment, the disclosure comprises a method for editing a nucleotide sequence in the genome of a cell, the method comprising introducing into said cell at least one polynucleotide modification template, at least one chimeric engineered guide RNA, and a Cas endonuclease comprising a first nuclease domain of SEQ ID NO: 88, and a second nuclease domain comprising at least one nuclease subdomain selected from the group consisting of SEQ ID NO: 90, SEQ ID NO: 92 and SEQ ID NO: 94, wherein said polynucleotide modification template comprises at least one nucleotide modification of said nucleotide sequence, wherein said chimeric engineered guide RNA and Cas endonuclease can form a complex that is capable of recognizing, binding to, and optionally nicking, cleaving, or covalently attaching to all or part of said target site.

The nucleotide to be edited can be located within or outside a target site recognized and cleaved by a Cas endonuclease. In one embodiment, the at least one nucleotide modification is not a modification at a target site recognized and cleaved by a Cas endonuclease. In another embodiment, there are at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the genomic target site.

The method for editing a nucleotide sequence in the genome of a cell can be a method without the use of an exogenous selectable marker by restoring function to a non-functional gene product as described in U.S. patent application 62/243,719, filed Oct. 20, 2015 and 62/309,033, filed Mar. 16, 2016.

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (for example by homologous recombination (HR), wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

Various methods and compositions can be employed to obtain a cell or organism having a polynucleotide of interest inserted in a target site for a Cas endonuclease. Such methods can employ homologous recombination (HR) to provide integration of the polynucleotide of Interest at the target site. In one method described herein, a polynucleotide of interest is introduced into the organism cell via a donor DNA construct. As used herein, “donor DNA” is a DNA construct that comprises a polynucleotide of interest to be inserted into the target site of a Cas endonuclease. The donor DNA construct can further comprise a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10: 957-963).

Episomal DNA molecules can also be ligated into the double-strand break, for example, integration of T-DNAs into chromosomal double-strand breaks (Chilton and Que, (2003) Plant Physiol 133:956-65; Salomon and Puchta, (1998) EMBO J 17:6086-95). Once the sequence around the double-strand breaks is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. PNAS (0027-8424), 111 (10), p. E924-E932).

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases such that the genomic region has sufficient homology to undergo homologous recombination with the corresponding region of homology.

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 8⁻¹%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some instances the regions of homology share significant sequence homology to the genomic sequence immediately flanking the target site, it is recognized that the regions of homology can be designed to have sufficient homology to regions that may be further 5′ or 3′ to the target site. The regions of homology can also have homology with a fragment of the target site along with downstream genomic regions

In one embodiment, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events: the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) Mol Cell Biol 4:2253-8; Ayares et al., (1986) Proc. Natl. Acad. Sci. USA 83:5199-203; Liskay et al., (1987) Genetics 115:161-7.

Alteration of the genome of a prokaryotic and eukaryotic cell or organism cell, for example, through homologous recombination (HR), is a powerful tool for genetic engineering. Homologous recombination has been demonstrated in plants (Halfter et al., (1992) Mol Gen Genet 231:186-93) and insects (Dray and Gloor, 1997, Genetics 147:689-99). Homologous recombination has also been accomplished in other organisms. For example, at least 150-200 bp of homology was required for homologous recombination in the parasitic protozoan Leishmania (Papadopoulou and Dumas, (1997) Nucleic Acids Res 25:4278-86). In the filamentous fungus Aspergillus nidulans, gene replacement has been accomplished with as little as 50 bp flanking homology (Chaveroche et al., (2000) Nucleic Acids Res 28:e97). Targeted gene replacement has also been demonstrated in the ciliate Tetrahymena thermophila (Gaertig et al., (1994) Nucleic Acids Res 22:5391-8). In mammals, homologous recombination has been most successful in the mouse using pluripotent embryonic stem cell lines (ES) that can be grown in culture, transformed, selected and introduced into a mouse embryo (Watson et al., 1992, Recombinant DNA, 2nd Ed., (Scientific American Books distributed by WH Freeman & Co.).

DNA double-strand breaks appear to be an effective factor to stimulate homologous recombination pathways (Puchta et al., (1995) Plant Mol Biol 28:281-92; Tzfira and White, (2005) Trends Biotechnol 23:567-9; Puchta, (2005) J Exp Bot 56:1-14). Using DNA-breaking agents, a two- to nine-fold increase of homologous recombination was observed between artificially constructed homologous DNA repeats in plants (Puchta et al., (1995) Plant Mol Biol 28:281-92). In maize protoplasts, experiments with linear DNA molecules demonstrated enhanced homologous recombination between plasmids (Lyznik et al., (1991) Mol Gen Genet 230:209-18).

In one embodiment, the disclosure comprises a method for modifying a target site in the genome of a cell, the method comprising providing to said cell at least one chimeric engineered guide RNA, at least one donor DNA, and a Cas endonuclease comprising a first nuclease domain of SEQ ID NO: 88, and a second nuclease domain comprising at least one nuclease subdomain selected from the group consisting of SEQ ID NO: 90, SEQ ID NO: 92 and SEQ ID NO: 94, wherein said at least one chimeric engineered guide RNA and Cas endonuclease can form a complex that is capable of recognizing, binding to, and optionally nicking, cleaving, or covalently attaching to all or part of said target site, wherein said donor DNA comprises a polynucleotide of interest. The method can further comprise identifying at least one cell that said polynucleotide of interest integrated in or near said target site.

Further uses for guide RNA/Cas endonuclease systems have been described (See U.S. Patent Application US 2015-0082478 A1, published on Mar. 19, 2015, WO2015/026886 A1, published on Feb. 26, 2015, US 2015-0059010 A1, published on Feb. 26, 2015, U.S. application 62/023,246, filed on Jul. 7, 2014, and U.S. application 62/036,652, filed on Aug. 13, 2014, all of which are incorporated by reference herein) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide a guide polynucleotide/Cas endonuclease complex to a unique DNA target site.

Polynucleotides of interest and/or traits can be stacked together in a complex trait locus as described in WO2012/129373, published Mar. 14, 2013, and in PCT/US13/22891, published Jan. 24, 2013, both hereby incorporated by reference. The guide polynucleotide/Lapis Cas endonuclease system described herein provides for an efficient system to generate double-strand breaks and allows for traits to be stacked in a complex trait locus.

A guide polynucleotide/Cas system as described herein, mediating gene targeting, can be used in methods for directing transgene insertion and/or for producing complex transgenic trait loci comprising multiple transgenes in a fashion similar as disclosed in WO2012/129373, published Mar. 14, 2013 where instead of using a double-strand break inducing agent to introduce a gene of interest, a guide polynucleotide/Cas system as disclosed herein is used. A complex trait locus includes a genomic locus that has multiple transgenes genetically linked to each other. By inserting independent transgenes within 0.1, 0.2, 0.3, 0.4, 0.5, 1.0, 2, or even 5 centimorgans (cM) from each other, the transgenes can be bred as a single genetic locus (see, for example, U.S. patent application Ser. No. 13/427,138) or PCT application PCT/US2012/030061. After selecting a plant comprising a transgene, plants containing (at least) one transgenes can be crossed to form an F1 that contains both transgenes. In progeny from these F1 (F2 or BC1) 1/500 progeny would have the two different transgenes recombined onto the same chromosome. The complex locus can then be bred as single genetic locus with both transgene traits. This process can be repeated to stack as many traits as desired.

The Cas endonuclease described herein (including the Lapis Cas) can be expressed and purified by methods known in the art (such as those described in Example 2 of U.S. patent applications 62/162,377 filed May 15, 2015, incorporated herein by reference).

Many endonucleases have been described to date that can recognize specific PAM sequences (see for example—U.S. patent applications 62/162,377 filed May 15, 2015 and 62/162,353 filed May 15, 2015 and Zetsche B et al. 2015. Cell 163, 1013) and cleave the target DNA at a specific positions. It is understood that based on the methods and embodiments described herein utilizing a novel guided Cas system one skilled in the art can now tailor these methods such that they can utilize any guided endonuclease system.

Endonucleases are enzymes that cleave the phosphodiester bond within a polynucleotide chain, and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H—N—H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. This cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

TAL effector nucleases are a class of sequence-specific nucleases that can be used to make double-strand breaks at specific target sequences in the genome of a plant or other organism. (Miller et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

Any one component of the guide polynucleotide/Cas endonuclease complex, the guide polynucleotide/Cas endonuclease complex itself, as well as the polynucleotide modification template(s) and/or donor DNA(s), can be introduced into a cell by any method known in the art.

“Introducing” is intended to mean presenting to the organism, such as a cell or organism, the polynucleotide or polypeptide or polynucleotide-protein complex, in such a manner that the component(s) gains access to the interior of a cell of the organism or to the cell itself. The methods and compositions do not depend on a particular method for introducing a sequence into an organism or cell, only that the polynucleotide or polypeptide gains access to the interior of at least one cell of the organism. Introducing includes reference to the incorporation of a nucleic acid into a eukaryotic or prokaryotic cell where the nucleic acid may be incorporated into the genome of the cell, and includes reference to the transient (direct) provision of a nucleic acid, protein or polynucleotide-protein complex (PGEN, RGEN) to the cell.

Methods for introducing polynucleotides or polypeptides or a polynucleotide-protein complex into cells or organisms are known in the art including, but not limited to, microinjection, electroporation, stable transformation methods, transient transformation methods, ballistic particle acceleration (particle bombardment), whiskers mediated transformation, Agrobacterium-mediated transformation, direct gene transfer, viral-mediated introduction, transfection, transduction, cell-penetrating peptides, mesoporous silica nanoparticle (MSN)-mediated direct protein delivery, topical applications, sexual crossing, sexual breeding, and any combination thereof.

For example, the guide polynucleotide can be introduced into a cell directly (transiently) as a single stranded or double stranded polynucleotide molecule. The guide RNA can also be introduced into a cell indirectly by introducing a recombinant DNA molecule comprising a heterologous nucleic acid fragment encoding the guide RNA, operably linked to a specific promoter that is capable of transcribing the guide RNA in said cell. The specific promoter can be, but is not limited to, a RNA polymerase Ill promoter, which allow for transcription of RNA with precisely defined, unmodified, 5′- and 3′-ends (Ma et al., 2014, Mol. Ther. Nucleic Acids 3:e161; DiCarlo et al., 2013, Nucleic Acids Res. 41: 4336-4343; WO2015026887, published on Feb. 26, 2015). Any promoter capable of transcribing the guide RNA in a cell can be used and includes a heat shock/heat inducible promoter operably linked to a nucleotide sequence encoding the guide RNA.

The Cas endonuclease can be introduced into a cell by directly introducing the Cas protein itself (referred to as direct delivery of Cas endonuclease), the mRNA encoding the Cas protein, and/or the guide polynucleotide/Cas endonuclease complex itself, using any method known in the art. The Cas endonuclease can also be introduced into a cell indirectly by introducing a recombinant DNA molecule that encodes the Cas endonuclease. The endonuclease can be introduced into a cell transiently or can be incorporated into the genome of the host cell using any method known in the art. Uptake of the endonuclease and/or the guided polynucleotide into the cell can be facilitated with a Cell Penetrating Peptide (CPP) as described in U.S. application 62/075,999, filed Nov. 6, 2014. Any promoter capable of expressing the Lapis Cas endonuclease in a cell can be used and includes a heat shock/heat inducible promoter operably linked to a nucleotide sequence encoding the Lapis Cas endonuclease.

Direct delivery of a polynucleotide modification template into plant cells can be achieved through particle mediated delivery, and any other direct method of delivery, such as but not limiting to, polyethylene glycol (PEG)-mediated transfection to protoplasts, whiskers mediated transformation, electroporation, particle bombardment, cell-penetrating peptides, or mesoporous silica nanoparticle (MSN)-mediated direct protein delivery can be successfully used for delivering a polynucleotide modification template in eukaryotic cells, such as plant cells.

The donor DNA can be introduced by any means known in the art. The donor DNA may be provided by any transformation method known in the art including, for example, Agrobacterium-mediated transformation or biolistic particle bombardment. The donor DNA may be present transiently in the cell or it could be introduced via a viral replicon. In the presence of the Cas endonuclease and the target site, the donor DNA is inserted into the transformed plant's genome.

Direct delivery of any one of the guided Cas system components can be accompanied by direct delivery (co-delivery) of other mRNAs that can promote the enrichment and/or visualization of cells receiving the guide polynucleotide/Cas endonuclease complex components. For example, direct co-delivery of the guide polynucleotide/Cas endonuclease components (and/or guide polynucleotide/Cas endonuclease complex itself) together with mRNA encoding phenotypic markers (such as but not limiting to transcriptional activators such as CRC (Bruce et al. 2000 The Plant Cell 12:65-79) can enable the selection and enrichment of cells without the use of an exogenous selectable marker by restoring function to a non-functional gene product as described in U.S. patent application 62/243,719, filed Oct. 20, 2015 and 62/309,033, filed Mar. 16, 2016.

Protocols for introducing polynucleotides, polypeptides or polynucleotide-protein complexes (PGEN, RGEN) into eukaryotic cells, such as plants or plant cells are known and include microinjection (Crossway et al., (1986) Biotechniques 4:320-34 and U.S. Pat. No. 6,300,543), meristem transformation (U.S. Pat. No. 5,736,369), electroporation (Riggs et al., (1986) Proc. Natl. Acad. Sci. USA 83:5602-6, Agrobacterium-mediated transformation (U.S. Pat. Nos. 5,563,055 and 5,981,840), whiskers mediated transformation (Ainley et al. 2013, Plant Biotechnology Journal 11:1126-1134; Shaheen A. and M. Arshad 2011 Properties and Applications of Silicon Carbide (2011), 345-358 Editor(s): Gerhardt, Rosario. Publisher: InTech, Rijeka, Croatia. CODEN: 69PQBP; ISBN: 978-953-307-201-2), direct gene transfer (Paszkowski et al., (1984) EMBO J 3:2717-22), and ballistic particle acceleration (U.S. Pat. Nos. 4,945,050; 5,879,918; 5,886,244; 5,932,782; Tomes et al., (1995) “Direct DNA Transfer into Intact Plant Cells via Microprojectile Bombardment” in Plant Cell, Tissue, and Organ Culture: Fundamental Methods, ed. Gamborg & Phillips (Springer-Verlag, Berlin); McCabe et al., (1988) Biotechnology 6:923-6; Weissinger et al., (1988) Ann Rev Genet 22:421-77; Sanford et al., (1987) Particulate Science and Technology 5:27-37 (onion); Christou et al., (1988) Plant Physiol 87:671-4 (soybean); Finer and McMullen, (1991) In Vitro Cell Dev Biol 27P:175-82 (soybean); Singh et al., (1998) Theor Appl Genet 96:319-24 (soybean); Datta et al., (1990) Biotechnology 8:736-40 (rice); Klein et al., (1988) Proc. Natl. Acad. Sci. USA 85:4305-9 (maize); Klein et al., (1988) Biotechnology 6:559-63 (maize); U.S. Pat. Nos. 5,240,855; 5,322,783 and 5,324,646; Klein et al., (1988) Plant Physiol 91:440-4 (maize); Fromm et al., (1990) Biotechnology 8:833-9 (maize); Hooykaas-Van Slogteren et al., (1984) Nature 311:763-4; U.S. Pat. No. 5,736,369 (cereals); Bytebier et al., (1987) Proc. Natl. Acad. Sci. USA 84:5345-9 (Liliaceae); De Wet et al., (1985) in The Experimental Manipulation of Ovule Tissues, ed. Chapman et al., (Longman, N.Y.), pp. 197-209 (pollen); Kaeppler et al., (1990) Plant Cell Rep 9:415-8) and Kaeppler et al., (1992) Theor Appl Genet 84:560-6 (whisker-mediated transformation); D'Halluin et al., (1992) Plant Cell 4:1495-505 (electroporation); Li et al., (1993) Plant Cell Rep 12:250-5; Christou and Ford (1995) Annals Botany 75:407-13 (rice) and Osjoda et al., (1996) Nat Biotechnol 14:745-50 (maize via Agrobacterium tumefaciens).

Alternatively, polynucleotides may be introduced into plant or plant cells by contacting cells or organisms with a virus or viral nucleic acids. Generally, such methods involve incorporating a polynucleotide within a viral DNA or RNA molecule. In some examples a polypeptide of interest may be initially synthesized as part of a viral polyprotein, which is later processed by proteolysis in vivo or in vitro to produce the desired recombinant protein. Methods for introducing polynucleotides into plants and expressing a protein encoded therein, involving viral DNA or RNA molecules, are known, see, for example, U.S. Pat. Nos. 5,889,191, 5,889,190, 5,866,785, 5,589,367 and 5,316,931.

The polynucleotide or recombinant DNA construct can be provided to or introduced into a prokaryotic and eukaryotic cell or organism using a variety of transient transformation methods. Such transient transformation methods include, but are not limited to, the introduction of the polynucleotide construct directly into the plant.

Nucleid acids and proteins can be provided to a cell by any method including methods using molecules to facilitate the uptake of anyone or all components of a guided Cas system (protein and/or nucleic acids), such as cell-penetrating peptides and nanocariers. See also US20110035836 Nanocarier based plant transfection and transduction, and EP 2821486 A1 Method of introducing nucleic acid into plant cells, incorporated herein by reference.

Other methods of introducing polynucleotides into a prokaryotic and eukaryotic cell or organism or plant part can be used, including plastid transformation methods, and the methods for introducing polynucleotides into tissues from seedlings or mature seeds.

Stable transformation is intended to mean that the nucleotide construct introduced into an organism integrates into a genome of the organism and is capable of being inherited by the progeny thereof. Transient transformation is intended to mean that a polynucleotide is introduced into the organism and does not integrate into a genome of the organism or a polypeptide is introduced into an organism. Transient transformation indicates that the introduced composition is only temporarily expressed or present in the organism.

Polynucleotides of interest are further described herein and include polynucleotides reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for genetic engineering will change accordingly.

Polynucleotides of interest include, but are not limited to, polynucleotides encoding important traits for agronomics, herbicide-resistance, insecticidal resistance, disease resistance, nematode resistance, herbicide resistance, microbial resistance, fungal resistance, viral resistance, fertility or sterility, grain characteristics, and commercial products.

General categories of polynucleotides of interest include, for example, genes of interest involved in information, such as zinc fingers, those involved in communication, such as kinases, and those involved in housekeeping, such as heat shock proteins. More specific polynucleotides of interest include, but are not limited to, genes involved in crop yield, grain quality, crop nutrient content, starch and carbohydrate quality and quantity as well as those affecting kernel size, sucrose loading, protein quality and quantity, nitrogen fixation and/or utilization, fatty acid and oil composition, genes encoding proteins conferring resistance to abiotic stress (such as drought, nitrogen, temperature, salinity, toxic metals or trace elements, or those conferring resistance to toxins such as pesticides and herbicides), genes encoding proteins conferring resistance to biotic stress (such as attacks by fungi, viruses, bacteria, insects, and nematodes, and development of diseases associated with these organisms).

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference.

Polynucleotides of interest include any nucleotide sequence encoding a protein or polypeptide that improves desirability of crops. Polynucleotide sequences of interest may encode proteins involved in providing disease or pest resistance. By “disease resistance” or “pest resistance” is intended that the plants avoid the harmful symptoms that are the outcome of the plant-pathogen interactions. Pest resistance genes may encode resistance to pests that have great yield drag such as rootworm, cutworm, European Corn Borer, and the like. Disease resistance and insect resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products. Genes encoding disease resistance traits include detoxification genes, such as against fumonisin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like. Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer period of time than cells that do not express the protein. Herbicide resistance traits may be introduced into plants by genes coding for resistance to herbicides that act to inhibit the action of acetolactate synthase (ALS, also referred to as acetohydroxyacid synthase, AHAS), in particular the sulfonylurea-type (UK: sulphonylurea) herbicides, genes coding for resistance to herbicides that act to inhibit the action of glutamine synthase, such as phosphinothricin or basta (e.g., the bar gene), glyphosate (e.g., the EPSP synthase gene and the GAT gene), HPPD inhibitors (e.g, the HPPD gene) or other such genes known in the art. See, for example, U.S. Pat. Nos. 7,626,077, 5,310,667, 5,866,775, 6,225,114, 6,248,876, 7,169,970, 6,867,293, and U.S. Provisional Application No. 61/401,456, each of which is herein incorporated by reference. The bar gene encodes resistance to the herbicide basta, the nptll gene encodes resistance to the antibiotics kanamycin and geneticin, and the ALS-gene mutants encode resistance to the herbicide chlorsulfuron.

Furthermore, it is recognized that the polynucleotide of interest may also comprise antisense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the polynucleotide of interest may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See, U.S. Pat. Nos. 5,283,184 and 5,034,323; herein incorporated by reference.

The polynucleotide of interest can also be a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that confer resistance to herbicidal compounds, such as sulphonylureas, glufosinate ammonium, bromoxynil, imidazolinones, and 2,4-dichlorophenoxyacetate (2,4-D). See for example, Acetolactase synthase (ALS) for resistance to sulfonylureas, imidazolinones, triazolopyrimidine sulfonamides, pyrimidinylsalicylates and sulphonylaminocarbonyl-triazolinones Shaner and Singh, 1997, Herbicide Activity: Toxicol Biochem Mol Biol 69-110); glyphosate resistant 5-enolpyruvylshikimate-3-phosphate (EPSPS)(Saroha et al. 1998, J. Plant Biochemistry & Biotechnology Vol 7:65-72);

Polynucleotides of interest includes genes that can be stacked or used in combination with other traits, such as but not limited to herbicide resistance or any other trait described herein. Polynucleotides of interest and/or traits can be stacked together in a complex trait locus as described in US-2013-0263324-A1, published 3 Oct. 2013 and in PCT/US13/22891, published Jan. 24, 2013, both applications are hereby incorporated by reference.

A polypeptide of interest includes any protein or polypeptide that is encoded by a polynucleotide of interest described herein.

Further provided are methods for identifying at least one plant cell, comprising in its genome, a polynucleotide of interest integrated at the target site. A variety of methods are available for identifying those plant cells with insertion into the genome at or near to the target site. Such methods can be viewed as directly analyzing a target sequence to detect any change in the target sequence, including but not limited to PCR methods, sequencing methods, nuclease digestion, Southern blots, and any combination thereof. See, for example, U.S. patent application Ser. No. 12/147,834, herein incorporated by reference to the extent necessary for the methods described herein. The method also comprises recovering a plant from the plant cell comprising a polynucleotide of Interest integrated into its genome. The plant may be sterile or fertile. It is recognized that any polynucleotide of interest can be provided, integrated into the plant genome at the target site, and expressed in a plant.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single- or double-stranded, optionally containing synthetic, non-natural, or altered nucleotide bases. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

“Open reading frame” is abbreviated ORF.

The terms “fragment that is functionally equivalent” and “functionally equivalent fragment” are used interchangeably herein. These terms refer to a portion or subsequence of an isolated nucleic acid fragment or polypeptide in which the ability to alter gene expression or produce a certain phenotype is retained whether or not the fragment encodes an active enzyme. For example, the fragment can be used in the design of genes to produce the desired phenotype in a modified plant. Genes can be designed for use in suppression by linking a nucleic acid fragment, whether or not it encodes an active enzyme, in the sense or antisense orientation relative to a plant promoter sequence.

The term “conserved domain” or “motif” means a set of amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

Polynucleotide and polypeptide sequences, variants thereof, and the structural relationships of these sequences can be described by the terms “homology”, “homologous”, “substantially identical”, “substantially similar” and “corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment.

Substantially similar nucleic acid sequences encompassed may be defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence in an in vitro hybridization assay. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salt(s)) at pH 7.0 to 8.3, and at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

“Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90% or 95%, or any integer percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, Wis.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, (1989) CABIOS 5:151-153; Higgins et al., (1992) Comput Appl Biosci 8:189-191) and found in the MegAlign™ program of the LASERGENE bioinformatics computing suite (DNASTAR Inc., Madison, Wis.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program.

The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign™ v6.1 program of the LASERGENE bioinformatics computing suite (DNASTAR Inc., Madison, Wis.). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

Unless otherwise stated, sequence identity/similarity values provided herein refer to the value obtained using GAP Version 10 (GCG, Accelrys, San Diego, Calif.) using the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89:10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases.

“BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence.

It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any integer percentage from 50% to 100%. Indeed, any integer amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

“Gene” includes a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in nature with its own regulatory sequences.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated plant is a plant comprising a mutated gene.

As used herein, a “targeted mutation” is a mutation in a gene (referred to as the target gene), including a native gene, that was made by altering a target sequence within the target gene using any method known to one skilled in the art, including a method involving a guided Cas endonuclease system as disclosed herein.

A guide polynucleotide/Cas endonuclease induced targeted mutation can occur in a nucleotide sequence that is located within or outside a genomic target site that is recognized and cleaved by the Cas endonuclease.

The term “genome” as it applies to a prokaryotic and eukaryotic cell or organism cells encompasses not only chromosomal DNA found within the nucleus, but organelle DNA found within subcellular components (e.g., mitochondria, or plastid) of the cell.

An “allele” is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “codon-modified gene” or “codon-preferred gene” or “codon-optimized gene” is a gene having its frequency of codon usage designed to mimic the frequency of preferred codon usage of the host cell.

“A plant-optimized nucleotide sequence” is a nucleotide sequence that has been optimized for expression in plants, particularly for increased expression in plants. A plant-optimized nucleotide sequence includes a codon-optimized gene. A plant-optimized nucleotide sequence can be synthesized by modifying a nucleotide sequence encoding a protein such as, for example, a Cas endonuclease as disclosed herein, using one or more plant-preferred codons for improved expression. See, for example, Campbell and Gowri (1990) Plant Physiol. 92:1-11 for a discussion of host-preferred codon usage.

“A mammalian-optimized nucleotide sequence” is a nucleotide sequence that has been optimized for expression in mammalian cells, particularly for increased expression in mammals. A mammalian-optimized nucleotide sequence includes a codon-optimized gene. A mammalian-optimized nucleotide sequence can be synthesized by modifying a nucleotide sequence encoding a protein such as, for example, a Cas endonuclease as disclosed herein, using one or more mammalian-preferred codons for improved expression.

Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference. Additional sequence modifications are known to enhance gene expression in a plant host. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given plant host, as calculated by reference to known genes expressed in the host plant cell. When possible, the sequence is modified to avoid one or more predicted hairpin secondary mRNA structures. Thus, “a plant-optimized nucleotide sequence” of the present disclosure comprises one or more of such sequence modifications.

A “promoter” is a region of DNA involved in recognition and binding of RNA polymerase and other proteins to initiate transcription. The promoter sequence consists of proximal and more distal upstream elements, the latter elements often referred to as enhancers. An “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters that cause a gene to be expressed in most cell types at most times are commonly referred to as “constitutive promoters”.

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters” if the promoters direct RNA synthesis preferably in certain tissues but also in other tissues at reduced levels.

A plant promoter includes a promoter capable of initiating transcription in a plant cell. For a review of plant promoters, see, Potenza et al., 2004, In Vitro Cell Dev Biol 40:1-22; Porto et al., 2014, Molecular Biotechnology (2014), 56(1), 38-49.

Constitutive promoters include, for example, the core CaMV 35S promoter (Odell et al., (1985) Nature 313:810-2); rice actin (McElroy et al., (1990) Plant Cell 2:163-71); ubiquitin (Christensen et al., (1989) Plant Mol Biol 12:619-32; ALS promoter (U.S. Pat. No. 5,659,026) and the like.

Tissue-preferred promoters can be utilized to target enhanced expression within a particular plant tissue. Tissue-preferred promoters include, for example, WO2013/103367 published on 11 Jul. 2013, Kawamata et al., (1997) Plant Cell Physiol 38:792-803; Hansen et al., (1997) Mol Gen Genet 254:337-43; Russell et al., (1997) Transgenic Res 6:157-68; Rinehart et al., (1996) Plant Physiol 112:1331-41; Van Camp et al., (1996) Plant Physiol 112:525-35; Canevascini et al., (1996) Plant Physiol 112:513-524; Lam, (1994) Results Probl Cell Differ 20:181-96; and Guevara-Garcia et al., (1993) Plant J 4:495-505. Leaf-preferred promoters include, for example, Yamamoto et al., (1997) Plant J 12:255-65; Kwon et al., (1994) Plant Physiol 105:357-67; Yamamoto et al., (1994) Plant Cell Physiol 35:773-8; Gotor et al., (1993) Plant J 3:509-18; Orozco et al., (1993) Plant Mol Biol 23:1129-38; Matsuoka et al., (1993) Proc. Natl. Acad. Sci. USA 90:9586-90; Simpson et al., (1958) EMBO J 4:2723-9; Timko et al., (1988) Nature 318:57-8. Root-preferred promoters include, for example, Hire et al., (1992) Plant Mol Biol 20:207-18 (soybean root-specific glutamine synthase gene); Miao et al., (1991) Plant Cell 3:11-22 (cytosolic glutamine synthase (GS)); Keller and Baumgartner, (1991) Plant Cell 3:1051-61 (root-specific control element in the GRP 1.8 gene of French bean); Sanger et al., (1990) Plant Mol Biol 14:433-43 (root-specific promoter of A. tumefaciens mannopine synthase (MAS)); Bogusz et al., (1990) Plant Cell 2:633-41 (root-specific promoters isolated from Parasponia andersonii and Trema tomentosa); Leach and Aoyagi, (1991) Plant Sci 79:69-76 (A. rhizogenes roIC and roID root-inducing genes); Teeri et al., (1989) EMBO J 8:343-50 (Agrobacterium wound-induced TR1′ and TR2′ genes); VfENOD-GRP3 gene promoter (Kuster et al., (1995) Plant Mol Biol 29:759-72); and rolB promoter (Capana et al., (1994) Plant Mol Biol 25:681-91; phaseolin gene (Murai et al., (1983) Science 23:476-82; Sengopta-Gopalen et al., (1988) Proc. Natl. Acad. Sci. USA 82:3320-4). See also, U.S. Pat. Nos. 5,837,876; 5,750,386; 5,633,363; 5,459,252; 5,401,836; 5,110,732 and 5,023,179.

Seed-preferred promoters include both seed-specific promoters active during seed development, as well as seed-germinating promoters active during seed germination. See, Thompson et al., (1989) BioEssays 10:108. Seed-preferred promoters include, but are not limited to, Cim1 (cytokinin-induced message); cZ19B1 (maize 19 kDa zein); and milps (myo-inositol-1-phosphate synthase); (WO00/11177; and U.S. Pat. No. 6,225,529). For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa gamma zein, waxy, shrunken 1, shrunken 2, globulin 1, oleosin, and nuc1. See also, WO00/12733, where seed-preferred promoters from END1 and END2 genes are disclosed.

The term “inducible promoter” refers to a promoter that selectively express a coding sequence or functional RNA in response to the presence of an endogenous or exogenous stimulus, for example by chemical compounds (chemical inducers) or in response to environmental, hormonal, chemical, and/or developmental signals. Inducible or regulated promoters include, for example, promoters induced or regulated by light, heat, stress, flooding or drought, salt stress, osmotic stress, phytohormones, wounding, or chemicals such as ethanol, abscisic acid (ABA), jasmonate, salicylic acid, or safeners.

Chemical inducible (regulated) promoters can be used to modulate the expression of a gene in a prokaryotic and eukaryotic cell or organism through the application of an exogenous chemical regulator. The promoter may be a chemical-inducible promoter, where application of the chemical induces gene expression, or a chemical-repressible promoter, where application of the chemical represses gene expression. Chemical-inducible promoters include, but are not limited to, the maize In2-2 promoter, activated by benzene sulfonamide herbicide safeners (De Veylder et al., (1997) Plant Cell Physiol 38:568-77), the maize GST promoter (GST-II-27, WO93/01294), activated by hydrophobic electrophilic compounds used as pre-emergent herbicides, and the tobacco PR-1a promoter (Ono et al., (2004) Biosci Biotechnol Biochem 68:803-7) activated by salicylic acid. Other chemical-regulated promoters include steroid-responsive promoters (see, for example, the glucocorticoid-inducible promoter (Schena et al., (1991) Proc. Natl. Acad. Sci. USA 88:10421-5; McNellis et al., (1998) Plant J 14:247-257); tetracycline-inducible and tetracycline-repressible promoters (Gatz et al., (1991) Mol Gen Genet 227:229-37; U.S. Pat. Nos. 5,814,618 and 5,789,156).

Pathogen inducible promoters induced following infection by a pathogen include, but are not limited to those regulating expression of PR proteins, SAR proteins, beta-1,3-glucanase, chitinase, etc.

A stress-inducible promoter includes the RD29A promoter (Kasuga et al. (1999) Nature Biotechnol. 17:287-91). One of ordinary skill in the art is familiar with protocols for simulating stress conditions such as drought, osmotic stress, salt stress and temperature stress and for evaluating stress tolerance of plants that have been subjected to simulated or naturally-occurring stress conditions.

Another example of an inducible promoter useful in plant cells, is the ZmCAS1 promoter, described in US patent application, US 2013-0312137A1, published on Nov. 21, 2013, incorporated by reference herein.

New promoters of various types useful in plant cells are constantly being discovered; numerous examples may be found in the compilation by Okamuro and Goldberg, (1989) In The Biochemistry of Plants, Vol. 115, Stumpf and Conn, eds (New York, N.Y.: Academic Press), pp. 1-82.

“Translation leader sequence” refers to a polynucleotide sequence located between the promoter sequence of a gene and the coding sequence. The translation leader sequence is present in the mRNA upstream of the translation start sequence. The translation leader sequence may affect processing of the primary transcript to mRNA, mRNA stability or translation efficiency. Examples of translation leader sequences have been described (e.g., Turner and Foster, (1995) Mol Biotechnol 3:225-236).

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. A RNA transcript is referred to as the mature RNA or mRNA when it is a RNA sequence derived from post-transcriptional processing of the primary transcript pre-mRNA. “Messenger RNA” or “mRNA” refers to the RNA that is without introns and that can be translated into protein by the cell. “cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

Standard recombinant DNA and molecular cloning techniques used herein are well known in the art and are described more fully in Sambrook et al., Molecular Cloning: A Laboratory Manual; Cold Spring Harbor Laboratory: Cold Spring Harbor, NY (1989). Transformation methods are well known to those skilled in the art and are described infra.

The term “recombinant” refers to an artificial combination of two otherwise separated segments of sequence, e.g., by chemical synthesis, or manipulation of isolated segments of nucleic acids by genetic engineering techniques.

The terms “plasmid”, “vector” and “cassette” refer to a linear or circular extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector containing a gene and having elements in addition to the gene that facilitates transformation of a particular host cell. “Expression cassette” refers to a specific vector containing a gene and having elements in addition to the gene that allow for expression of that gene in a host.

The terms “recombinant DNA molecule”, “recombinant DNA construct”, “expression construct”, “construct”, and “recombinant construct” are used interchangeably herein. A recombinant DNA construct comprises an artificial combination of nucleic acid sequences, e.g., regulatory and coding sequences that are not all found together in nature. For example, a recombinant DNA construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to introduce the vector into the host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:2411-2418; De Almeida et al., (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The term “expression”, as used herein, refers to the production of a functional end-product (e.g., an mRNA, guide RNA, or a protein) in either precursor or mature form.

“Mature” protein refers to a post-translationally processed polypeptide (i.e., one from which any pre- or propeptides present in the primary translation product have been removed). “Precursor” protein refers to the primary product of translation of mRNA (i.e., with pre- and propeptides still present). Pre- and propeptides may be but are not limited to intracellular localization signals.

The presently disclosed guide polynucleotides, Cas endonucleases, polynucleotide modification templates, donor DNAs, guide polynucleotide/Cas endonuclease systems and any one combination thereof, can be introduced into a cell.

Cells include, but are not limited to, mammalian, human, non-human, animal, bacterial, fungal, insect, yeast, non-conventional yeast, and plant cells as well as plants and seeds produced by the methods described herein.

Any plant or plant part can be used, including monocot and dicot plants or plant part.

Examples of monocot plants that can be used include, but are not limited to, corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

The term “dicotyledonous” or “dicot” refers to the subclass of angiosperm plants also knows as “dicotyledoneae” and includes reference to whole plants, plant organs (e.g., leaves, stems, roots, etc.), seeds, plant cells, and progeny of the same. Examples of dicot plants that can be used include, but are not limited to, soybean (Glycine max), Brassica species (Canola) (Brassica napus, B. campestris, Brassica rapa, Brassica. juncea), alfalfa (Medicago sativa),), alfalfa (Medicago sativa), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), potato (Solanum tuberosum.

Plant that can be used include safflower (Carthamus tinctorius), sweet potato (Ipomoea batatus), cassava (Manihot esculenta), coffee (Coffea spp.), coconut (Cocos nucifera), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis).

The term “plant” includes whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. Plant parts include differentiated and undifferentiated tissues including, but not limited to roots, stems, shoots, leaves, pollens, seeds, tumor tissue and various forms of cells and culture (e.g., single cells, protoplasts, embryos, and callus tissue). The plant tissue may be in plant or in a plant organ, tissue or cell culture. The term “plant organ” refers to plant tissue or a group of tissues that constitute a morphologically and functionally distinct part of a plant. The term “genome” refers to the entire complement of genetic material (genes and non-coding sequences) that is present in each cell of an organism, or virus or organelle; and/or a complete set of chromosomes inherited as a (haploid) unit from one parent. “Progeny” comprises any subsequent generation of a plant.

As used herein, the term “plant part” refers to plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

A transgenic plant includes, for example, a plant which comprises within its genome a heterologous polynucleotide introduced by a transformation step. The heterologous polynucleotide can be stably integrated within the genome such that the polynucleotide is passed on to successive generations. The heterologous polynucleotide may be integrated into the genome alone or as part of a recombinant DNA construct. A transgenic plant can also comprise more than one heterologous polynucleotide within its genome. Each heterologous polynucleotide may confer a different trait to the transgenic plant. A heterologous polynucleotide can include a sequence that originates from a foreign species, or, if from the same species, can be substantially modified from its native form. Transgenic can include any cell, cell line, callus, tissue, plant part or plant, the genotype of which has been altered by the presence of heterologous nucleic acid including those transgenics initially so altered as well as those created by sexual crosses or asexual propagation from the initial transgenic. The alterations of the genome (chromosomal or extra-chromosomal) by conventional plant breeding methods, by the genome editing procedure described herein that does not result in an insertion of a foreign polynucleotide, or by naturally occurring events such as random cross-fertilization, non-recombinant viral infection, non-recombinant bacterial transformation, non-recombinant transposition, or spontaneous mutation are not intended to be regarded as transgenic.

In certain embodiments of the disclosure, a fertile plant is a plant that produces viable male and female gametes and is self-fertile. Such a self-fertile plant can produce a progeny plant without the contribution from any other plant of a gamete and the genetic material contained therein. Other embodiments of the disclosure can involve the use of a plant that is not self-fertile because the plant does not produce male gametes, or female gametes, or both, that are viable or otherwise capable of fertilization. As used herein, a “male sterile plant” is a plant that does not produce male gametes that are viable or otherwise capable of fertilization. As used herein, a “female sterile plant” is a plant that does not produce female gametes that are viable or otherwise capable of fertilization. It is recognized that male-sterile and female-sterile plants can be female-fertile and male-fertile, respectively. It is further recognized that a male fertile (but female sterile) plant can produce viable progeny when crossed with a female fertile plant and that a female fertile (but male sterile) plant can produce viable progeny when crossed with a male fertile plant.

The term “non-conventional yeast” herein refers to any yeast that is not a Saccharomyces (e.g., S. cerevisiae) or Schizosaccharomyces yeast species. (see Non-Conventional Yeasts in Genetics, Biochemistry and Biotechnology: Practical Protocols” (K. Wolf, K. D. Breunig, G. Barth, Eds., Springer-Verlag, Berlin, Germany, 2003).

The term “crossed” or “cross” or “crossing” in the context of this disclosure means the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

The term “introgression” refers to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene, a modified (mutated or edited) native allele, or a selected allele of a marker or QTL.

A “centimorgan” (cM) or “map unit” is the distance between two linked genes, markers, target sites, loci, or any pair thereof, wherein 1% of the products of meiosis are recombinant. Thus, a centimorgan is equivalent to a distance equal to a 1% average recombination frequency between the two linked genes, markers, target sites, loci, or any pair thereof.

The present disclosure finds use in the breeding of plants comprising one or more introduced traits.

Maize plants (Zea mays L.) can be bred by both self-pollination and cross-pollination techniques. Maize has male flowers, located on the tassel, and female flowers, located on the ear, on the same plant. It can self-pollinate (“selfing”) or cross pollinate. Natural pollination occurs in maize when wind blows pollen from the tassels to the silks that protrude from the tops of the incipient ears. Pollination may be readily controlled by techniques known to those of skill in the art. The development of maize hybrids requires the development of homozygous inbred lines, the crossing of these lines, and the evaluation of the crosses. Pedigree breeding and recurrent selections are two of the breeding methods used to develop inbred lines from populations. Breeding programs combine desirable traits from two or more inbred lines or various broad-based sources into breeding pools from which new inbred lines are developed by selfing and selection of desired phenotypes. A hybrid maize variety is the cross of two such inbred lines, each of which may have one or more desirable characteristics lacked by the other or which complement the other. The new inbreds are crossed with other inbred lines and the hybrids from these crosses are evaluated to determine which have commercial potential. The hybrid progeny of the first generation is designated F1. The F1 hybrid is more vigorous than its inbred parents. This hybrid vigor, or heterosis, can be manifested in many ways, including increased vegetative growth and increased yield.

Hybrid maize seed can be produced by a male sterility system incorporating manual detasseling. To produce hybrid seed, the male tassel is removed from the growing female inbred parent, which can be planted in various alternating row patterns with the male inbred parent. Consequently, providing that there is sufficient isolation from sources of foreign maize pollen, the ears of the female inbred will be fertilized only with pollen from the male inbred. The resulting seed is therefore hybrid (F1) and will form hybrid plants.

Field variation impacting plant development can result in plants tasseling after manual detasseling of the female parent is completed. Or, a female inbred plant tassel may not be completely removed during the detasseling process. In any event, the result is that the female plant will successfully shed pollen and some female plants will be self-pollinated. This will result in seed of the female inbred being harvested along with the hybrid seed which is normally produced. Female inbred seed does not exhibit heterosis and therefore is not as productive as F1 seed. In addition, the presence of female inbred seed can represent a germplasm security risk for the company producing the hybrid.

Alternatively, the female inbred can be mechanically detasseled by machine. Mechanical detasseling is approximately as reliable as hand detasseling, but is faster and less costly. However, most detasseling machines produce more damage to the plants than hand detasseling. Thus, no form of detasseling is presently entirely satisfactory, and a need continues to exist for alternatives which further reduce production costs and to eliminate self-pollination of the female parent in the production of hybrid seed.

Mutations that cause male sterility in plants have the potential to be useful in methods for hybrid seed production for crop plants such as maize and can lower production costs by eliminating the need for the labor-intensive removal of male flowers (also known as de-tasseling) from the maternal parent plants used as a hybrid parent. Examples of genes used in such ways include male fertility genes such as MS26 (see for example U.S. Pat. Nos. 7,098,388, 7,517,975, 7,612,251), MS45 (see for example U.S. Pat. Nos. 5,478,369, 6,265,640) or MSCA1 (see for example U.S. Pat. No. 7,919,676).

Mutations that cause male sterility in maize have been produced by a variety of methods such as X-rays or UV-irradiations, chemical treatments, or transposable element insertions (ms23, ms25, ms26, ms32) (Chaubal et al. (2000) Am J Bot 87:1193-1201). Conditional regulation of fertility genes through fertility/sterility “molecular switches” could enhance the options for designing new male-sterility systems for crop improvement (Unger et al. (2002) Transgenic Res 11:455-465).

Chromosomal intervals that correlate with a phenotype or trait of interest can be identified. A variety of methods well known in the art are available for identifying chromosomal intervals. The boundaries of such chromosomal intervals are drawn to encompass markers that will be linked to the gene controlling the trait of interest. In other words, the chromosomal interval is drawn such that any marker that lies within that interval (including the terminal markers that define the boundaries of the interval) can be used as a marker for northern leaf blight resistance. In one embodiment, the chromosomal interval comprises at least one QTL, and furthermore, may indeed comprise more than one QTL. Close proximity of multiple QTLs in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identifies the same QTL or two different QTL. The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window.

A variety of methods are available to identify those cells having an altered genome at or near a target site without using a screenable marker phenotype. Such methods can be viewed as directly analyzing a target sequence to detect any change in the target sequence, including but not limited to PCR methods, sequencing methods, nuclease digestion, Southern blots, and any combination thereof.

Proteins may be altered in various ways including amino acid substitutions, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates containing target sites.

Standard DNA isolation, purification, molecular cloning, vector construction, and verification/characterization methods are well established, see, for example Sambrook et al., (1989) Molecular Cloning: A Laboratory Manual, (Cold Spring Harbor Laboratory Press, NY). Vectors and constructs include circular plasmids, and linear polynucleotides, comprising a polynucleotide of interest and optionally other components including linkers, adapters, regulatory or analysis. In some examples a recognition site and/or target site can be contained within an intron, coding sequence, 5′ UTRs, 3′ UTRs, and/or regulatory regions.

The meaning of abbreviations is as follows: “sec” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” mean micromole(s), “g” means gram(s), “μg” means microgram(s), “ng” means nanogram(s), “U” means unit(s), “bp” means base pair(s) and “kb” means kilobase(s).

Non-limiting examples of compositions and methods disclosed herein are as follows:

1. A guide RNA/Cas endonuclease complex comprising at least one guide RNA and a Cas endonuclease, wherein said Cas endonuclease comprises a first nuclease domain of SEQ ID NO: 88, and a second nuclease domain comprising at least one nuclease subdomain selected from the group consisting of SEQ ID NO: 90, SEQ ID NO: 92 and SEQ ID NO: 94, wherein said guide RNA is a chimeric engineered guide RNA, wherein said guide RNA/Cas endonuclease complex is capable of recognizing, binding to, and optionally nicking, cleaving, or covalently attaching to all or part of a target sequence.
2. The guide RNA/Cas endonuclease complex of embodiment 1, wherein said Cas endonuclease has at least 80% sequence identity to SEQ ID NO: 1.
3. The guide RNA/Cas endonuclease complex of embodiments 1-2 comprising at least one chimeric engineered guide RNA comprising a variable targeting domain that can recognize a target DNA in a eukaryotic cell.
4. The guide RNA/Cas endonuclease complex of embodiments 1-2, wherein said target sequence is located in the genome of a prokaryotic or eukaryotic cell.
5. A method for modifying a target site in the genome of a cell, the method comprising introducing into said cell at least one chimeric engineered guide RNA, and a Cas endonuclease comprising a first nuclease domain of SEQ ID NO: 88, and a second nuclease domain comprising at least one nuclease sub-domain selected from the group consisting of SEQ ID NO: 90, SEQ ID NO: 92 and SEQ ID NO: 94, wherein said chimeric engineered guide RNA and Cas endonuclease can form a complex that is capable of recognizing, binding to, and optionally nicking, cleaving, or covalently attaching to all or part of said target site.
6. The method of embodiment 5, further comprising identifying at least one cell that has a modification at said target, wherein the modification at said target site is selected from the group consisting of (i) a replacement of at least one nucleotide, (ii) a deletion of at least one nucleotide, (iii) an insertion of at least one nucleotide, and (iv) any combination of (i)-(iii).
7. A method for editing a nucleotide sequence in the genome of a cell, the method comprising introducing into said cell at least one polynucleotide modification template, at least one chimeric engineered guide RNA, and a Cas endonuclease comprising a first nuclease domain of SEQ ID NO: 88, and a second nuclease domain comprising at least one nuclease sub-domain selected from the group consisting of SEQ ID NO: 90, SEQ ID NO: 92 and SEQ ID NO: 94, wherein said polynucleotide modification template comprises at least one nucleotide modification of said nucleotide sequence, wherein said chimeric engineered guide RNA and Cas endonuclease can form a complex that is capable of recognizing, binding to, and optionally nicking, cleaving, or covalently attaching to all or part of said target site.
8. A method for modifying a target site in the genome of a cell, the method comprising providing to said cell at least one chimeric engineered guide RNA, at least one donor DNA, and a Cas endonuclease comprising a first nuclease domain of SEQ ID NO: 88, and a second nuclease domain comprising at least one nuclease sub-domain selected from the group consisting of SEQ ID NO: 90, SEQ ID NO: 92 and SEQ ID NO: 94, wherein said at least one chimeric engineered guide RNA and Cas endonuclease can form a complex that is capable of recognizing, binding to, and optionally nicking, cleaving, or covalently attaching to all or part of said target site, wherein said donor DNA comprises a polynucleotide of interest.
9. The method of embodiment 8, further comprising identifying at least one cell that said polynucleotide of interest integrated in or near said target site.
10. The method of any one of embodiments 5-9, wherein the cell is selected from the group consisting of a prokaryotic or eukaryotic cell.
11. The method of any one of embodiments 5-9, wherein the cell is selected from the group consisting of a mammalian, human cell, non-human cell, animal cell, bacterial cell, fungal cell, insect cell, yeast cell, non-conventional yeast cell, and a plant cell.
12. The method of embodiment 11, wherein the plant cell is selected from the group consisting of a monocot and dicot cell.
13. The method of embodiment 12 wherein the plant cell is selected from the group consisting of maize, rice, sorghum, rye, barley, wheat, millet, oats, sugarcane, turfgrass, or switchgrass, soybean, canola, alfalfa, sunflower, cotton, tobacco, peanut, potato, tobacco, Arabidopsis, and safflower cell.
14. A plant comprising a modified target site, wherein said plant originates from a plant cell comprising a modified target site produced by the method of any one of embodiments 5-6.
15. A plant comprising an edited nucleotide, wherein said plant originates from a plant cell comprising an edited nucleotide produced by the method of embodiment 7.
16. A plant comprising a polynucleotide of interest, wherein said plant originates from a plant cell comprising a polynucleotide of interest produced by the method of any one of embodiments 8-9.
17. A recombinant DNA polynucleotide comprising a promoter operably linked to a plant-optimized polynucleotide encoding a Cas endonuclease, wherein said Cas endonuclease comprises a first nuclease domain of SEQ ID NO: 88, and a second nuclease domain comprising at least one nuclease sub-domain selected from the group consisting of SEQ ID NO: 90, SEQ ID NO: 92 and SEQ ID NO: 94.
18. A kit for binding, cleaving or nicking a target sequence in a prokaryotic or eukaryotic cell or organism, said kit comprising a guide polynucleotide specific for said target sequence, and a Cas endonuclease or a polynucleotide encoding said Cas endonuclease, wherein said Cas endonuclease comprises a first nuclease domain of SEQ ID NO: 88, and a second nuclease domain comprising at least one nuclease sub-domain selected from the group consisting of SEQ ID NO: 90, SEQ ID NO: 92 and SEQ ID NO: 94, wherein said guide polynucleotide is capable of forming a guide polynucleotide/Cas endonuclease complex, wherein said complex can recognize, bind to, and optionally nick or cleave said target sequence.
19. A guide RNA/Cas endonuclease complex comprising at least one chimeric engineered guide RNA and a Cas endonuclease, wherein said Cas endonuclease comprises a first nuclease domain of SEQ ID NO: 88 or a functional fragment of SEQ ID NO: 88, and a second nuclease domain comprising at least one nuclease sub-domain selected from the group consisting of SEQ ID NO: 90, SEQ ID NO: 92 and SEQ ID NO: 94, or a functional fragment of said second domain, wherein said guide RNA/Cas endonuclease complex is capable of recognizing, binding to, and optionally nicking, cleaving, or covalently attaching to all or part of a target sequence.
20. A guide RNA/Cas endonuclease complex comprising at least one chimeric engineered guide RNA and a Cas endonuclease comprising at least one nuclease domain or subdomain selected from the group consisting of SEQ ID NO: 88, wherein said guide RNA/Cas endonuclease complex is capable of recognizing, binding to, and optionally nicking, cleaving, or covalently attaching to all or part of a target sequence.
21. A guide RNA/Cas endonuclease complex comprising a Cas endonuclease of SEQ ID NO: 1, or a functional fragment thereof, and at least one chimeric engineered guide RNA, wherein said guide RNA/Cas endonuclease complex is capable of recognizing, binding to, and optionally nicking, cleaving, or covalently attaching to all or part of a target sequence
22. A guide RNA/Cas endonuclease complex comprising at least one chimeric engineered guide RNA and a Cas endonuclease, wherein said Cas endonuclease is encoded by a codon optimized sequence of SEQ ID NO: 97, wherein said guide RNA/Cas endonuclease complex is capable of recognizing, binding to, and optionally nicking, cleaving, or covalently attaching to all or part of a target sequence.
23. A recombinant DNA polynucleotide comprising a promoter operably linked to a plant-optimized polynucleotide encoding a Lapis Cas endonuclease.
24. A kit for binding, cleaving or nicking a target sequence in a plant cell or plant, said kit comprising a guide polynucleotide specific for said target sequence, and a Lapis Cas endonuclease or a plant-optimized polynucleotide encoding a Lapis Cas endonuclease, wherein said guide polynucleotide is capable of forming a guide polynucleotide/Lapis Cas endonuclease complex, wherein said complex can recognize, bind to, and optionally nick or cleave said target sequence.
25. A chimeric engineered guide RNA capable of forming a guide RNA/Cas endonuclease complex that can recognize, bind to, and optionally nick, cleave, or covalently attach to a target sequence, wherein said guide RNA is selected from the group consisting of SEQ ID NOs: 128-138.

### EXAMPLES

In the following Examples, unless otherwise stated, parts and percentages are by weight and degrees are Celsius. It should be understood that these Examples, while indicating embodiments of the disclosure, are given by way of illustration only. From the above discussion and these Examples, one skilled in the art can make various changes and modifications of the disclosure to adapt it to various usages and conditions. Such modifications are also intended to fall within the scope of the appended claims.

### Example 1

### Identification of CRISPR Associated (Cas) Endonucleases with Novel Cleavage Domains

Cas endonucleases with novel cleavage domains were identified by first searching for the presence of clustered regularly interspaced short palindromic repeats (CRISPRs) indicative of the CRISPR-Cas nucleic acid based adaptive immune systems of bacteria and archaea (Bhaya et al. (2011) Annual Review Genetics, 45:273-297; Wiedenheft et al. (2012) Nature, 482:331-338) using PILER-CR (Edgar R. (2007) BMC Bioinformatics 8:18). Next, the DNA regions surrounding the CRISPR array (about 20 kb 5 prime and 3 prime of the CRISPR array) were examined for the presence of open-reading frames (ORFs) encoding proteins greater than 500 amino acids. Next, to identify CRISPR associated genes encoding homology to Cas9 endonucleases, multiple sequence alignment of protein sequences from a diverse collection of Cas9 endonucleases was performed using MUSCLE (Edgar R. (2004) Nucleic Acids Research, 32(5): 1792-97). The alignments were examined, curated and used to build profile hidden Markov models (HMM) for Cas9 sub-families using HMMER (Eddy S. R. (1998) Bioinformatics, 14:755-763; Eddy S. R. (2011) PLoS Comp. Biol., 7:e1002195). The resulting HMM models were then utilized to search protein sequences translated from the CRISPR associated ORFs for the presence of cas genes with homology to Cas9. Once identified, the resulting proteins were then examined for the presence of novel cleavage domains. This was accomplished by searching for the absence of a HNH or related DNA cleavage domains (e.g. cysHNH, HNN, or cysHNN) as defined in Kuhlmann et al. (1999) FEBS Letters 463:1-2, Aravind et al. (2000) Nucleic Acid Research 28:3417-3432, Mate et al. (2004) The Journal of Biological Chemistry 279:34763-34769, Keeble et al. (2005) Nucleic Acids and Molecular Biology 16:49-65, Biertumpfel et al. (2007) Nature 449:616-620 and Nishimasu et al. (2014) Cell 156:935-949 or RuvC or related DNA cleavage domains as defined in Ariyoshi et al. (1994) Cell 78:1063-1072, Aravind et al. (2000) and Nishimasu et al. (2014).

Interestingly, our search revealed the presence of a novel Cas protein from Lactobacillus apis (referred herein as Lapis) lacking the signature residues of a HNH endonuclease domain (see Kuhlmann et al. (1999), Aravind et al. (2000), Mate et al. (2004), Keeble et al. (2005), Biertumpfel et al. (2007) and Nishimasu et al. (2014) for descriptions of signature HNH domains). An alignment of the novel protein sequence of Lapis (referred herein as Lapis Cas; SEQ ID NO: 1) with a collection of 86 diverse Cas9 proteins whose phylogenetic relationship were reported in Fonfara et al. (2014) Nucleic Acids Research 42:2577-2590 (SEQ ID NO: 2-87), revealed that the key domain involved in the catalytic activity of the HNH motif of other Cas9 proteins was significantly different than the corresponding region in the Lapis protein (FIG. 1). Specifically, the Cas protein from Lapis (SEQ ID NO: 1) lacked the absolutely conserved histidine residue (H, His) which serves as a base to activate a water molecule for catalysis and is foundational to the cleavage mechanism of all HNH domains (Kuhlmann et al. (1999), Aravind et al. (2000), Mate et al. (2004), Keeble et al. (2005), Biertumpfel et al. (2007) and Nishimasu et al. (2014)) (FIG. 1). Additionally, the aspartic acid (D, Asp) residue typical for Cas9 endonucleases (for example Asp839 in Streptococcus pyogenes (Spy) Cas9 (SEQ ID NO: 2) which helps to coordinate a metal ion has been substituted with an arginine (ft Arg) (FIG. 1). Taken together, this analysis and results indicate that the Cas protein of Lactobacillus apis (Lapis) identified herein (referred to as LapisCas) does not contain an HNH nucleolytic domain, and instead contains a novel protein cleavage domain (SEQ ID NO: 88).

Analysis of the RuvC cleavage domain typical for Cas9 endonucleases (Gasiunas et al., 2012, Proc Natl Acad Sci USA. 109:E2579-2586; Jinek et al., 2012, Science. 337: 816-821; and Nishimasu et al., 2014 Cell, 156(5):935-949) in the Lactobacillus apis Cas (Lapis Cas) protein likewise revealed modifications that place it outside the definition of a RuvC cleavage domain (Ariyoshi et al. (1994), Aravind et al. 2000 and Nishimasu et al. (2014)). As shown in FIGS. 2-4, the most striking changes in the Lapis Cas protein when compared to the Cas9 RuvC domain were alterations in the acidic residues that traditionally co-coordinate metal ions responsible for facilitating DNA cleavage. As demonstrated herein (FIGS. 2 and 3), two of the four key residues typical for a RuvC domain, an aspartic acid (D, Asp) (for example Asp10 in Spy Cas9 (SEQ ID NO:2)) and a glutamic acid (E, Glu) (for example Glu762 in Spy Cas9 (SEQ ID NO:2)) which is conserved in RuvC domains of all other proteins (Ariyoshi et al. (1994) and Aravind et al. 2000), were replaced by serines (S, Ser) in the Lapis Cas protein (Ser13 and Ser799; SEQ ID NO:1). Additionally, a histidine residue present in most other Cas9s (for example His983 in Spy Cas9 (SEQ ID NO: 2)) has been replaced by an asparagine (N, Asn) in the Lapis Cas protein (FIG. 4). Taken together, this suggests that the Lapis Cas protein identified herein does not contain an RuvC nucleolytic domain and instead contains novel protein cleavage domains (SEQ ID NOs: 90, 92 and 94).

Next, the CRISPR-Cas locus architecture containing the novel Cas protein from Lactobacillus apis (Lapis) was examined as described below. The presence or absence of cas1 and cas2 genes were confirmed by comparing protein translations of ORFs 201 nucleotides within the CRISPR-Cas locus of Lapis (SEQ ID NO: 96) against the NCBI protein database for those matching known Cas1 and Cas2 proteins using the PSI-BLAST program (Altschul S F, et al. (1997) Nucleic Acids Res. 25:3389-3402). Additional CRISPR repeats missed by the PILER-CR program (Edgar R. (2007)) were identified by performing pairwise alignments of the locus with the CRISPR array repeat consensus (Altschul S F, et al. (1997)). The putative tracrRNA encoding region, termed the anti-repeat, was established by searching the locus for regions (distinct from the CRISPR array) with complete to partial homology to the repeats in the CRISPR array (as described in U.S. patent applications 62/162,377 filed May 15, 2015, 62/162,353 filed May 15, 2015 and 62/196,535 filed Jul. 24, 2015, all three applications incorporated in their entirety herein by reference). Once the anti-repeat was identified, the possible transcriptional directions of the tracrRNA was considered by examining the secondary structures and possible termination signals present in a RNA version of the sense and anti-sense genomic DNA sequences surrounding the anti-repeat (as described in U.S. patent applications 62/162,377 filed May 15, 2015, 62/162,353 filed May 15, 2015 and 62/196,535 filed Jul. 24, 2015, all three applications incorporated in their entirety herein by reference).

Surprisingly, no cas1 and cas2 genes were identified in the proximity of the Lapis cas gene distinguishing it from Type II CRISPR-Cas systems being minimally comprised of a tracrRNA encoding region, a cas9 gene, a cas1 gene, a cas2 gene, and a CRISPR array (Makarova et al. (2015) Nat. Rev. Microbiol. 13:722-736) (FIG. 5). Additionally, the Lapis CRISPR array identified herein is apparently organized into 6 dispersed groupings with the tracrRNA-like encoding region being duplicated immediately 5 prime of each array (FIG. 5). Furthermore, five additional copies of the tracrRNA-like encoding region were found 5 prime of the dispersed CRISPR (dCRISPR) arrays (FIG. 5). Taken together, the CRISPR-Cas locus architecture surrounding the Lapis cas gene identified herein indicates that it is a new previously undefined type of CRISPR-Cas system.

The genomic DNA sequence and length of the Lapis cas gene ORF and cas gene translation (not including the stop codon) are referenced in Table 2. Table 3 lists the consensus sequence of the CRISPR array repeats and the sequence of the putative tracrRNA encoding regions (as DNA sequence on the same strand as the cas gene ORF).

TABLE 2
Sequence and length of the Cas gene ORF and Cas gene translation
from Lactobacillus apis CRISPR-Cas system identified herein.

| | | Translation of Cas | |
|---|---|---|---|
| Cas Gene | | Gene ORF (not | |
| ORF | | including the | Length of Cas Gene |
| (SEQ ID | Length of Cas | stop codon) | Translation (No. of |
| NO) | Gene ORF (bp) | (SEQ ID NO) | Amino Acids) |
| 97 | 4173 | 1 | 1391 |

TABLE 3
CRISPR repeat consensus and putative tracrRNA encoding regions
from the Lactobacillus apis CRISPR-Cas system identified herein.

| CRISPR repeat | Putative tracrRNA |
|---|---|
| consensus | sequences |
| (SEQ ID NO) | (SEQ ID NO) |
| 98 | 99-109 |

Rapid in vitro methods to characterize the protospacer adjacent motif (PAM) requirement of Cas proteins have been described (see PCT/US16/32073 filed May 12, 2016, PCT/US16/32028 filed May 12, 2016, incorporated in their entirety herein by reference) and can be used to characterize the PAM preference of the novel CRISPR-Cas systems described herein. Once a guide RNA or guide RNAs that supports cleavage has been established, the PAM specificity of each Cas endonuclease can be assayed (as described in Examples 7, 14 and 15 U.S. patent application 62/162,377 filed May 15, 2015). After PAM preferences have been determined, the sgRNAs may be further refined for maximal activity or cellular transcription by either increasing or decreasing the tracrRNA 3′ end tail length, increasing or decreasing crRNA repeat and tracrRNA anti-repeat length, modifying the 4 nt self-folding loop or altering the sequence composition.

Following characterization of the guide RNA and PAM sequence, the Cas endonuclease and guide polynucleotide(s) may be optimized for maximal expression and nuclear localization in eukaryotic cells (as described in Example 12 of PCT/US16/32073, published May 12, 2016) or delivered directly as Cas protein guide polynucleotide complexes (as described in U.S. patent application 62/243,719, filed Oct. 20, 2015 and 62/309,033, filed Mar. 16, 2016) to cleave, nick or bind desired target sites.

### Example 2

### Lactobacillus apis CRISPR-Cas System Utilizes Single Naturally Occurring RNAs to Direct Target Recognition

A guide RNA or guide RNAs capable of directing Lactobacillus apis (Lapis) target recognition were first determined by computational inspection of the regions encoding the putative trans-activating CRISPR RNAs (tracrRNAs) (SEQ ID NOs: 99-109) in the Lapis CRISPR-Cas locus (FIG. 5). Interestingly, when the putative tracrRNA regions (SEQ ID NOs: 99-109) were simulated to be transcribed in the anti-sense direction relative to the Lapis cas gene (SEQ ID NOs: 110-120) and examined with an RNA folding algorithm (UNAfold (Markham and Zuker (2008) Methods Mol Biol. 453:3-31)), they exhibited an unusual secondary structure near the 5 prime end of their sequence (FIGS. 6-16). Curiously, these structures were reminiscent of that observed for an engineered single guide RNA (sgRNA), a non-natural chimeric fusion of a CRISPR RNA (crRNA) and tracrRNA (Jinek et al. (2012) and Briner et al. (2014) Mol. Cell. 56:333-339). This included a CRISPR repeat-like sequence, a loop sequence that promoted self-folding, an anti-repeat-like sequence with partial complementation to the repeat sequence, and a 3 prime region with tracrRNA hairpin-like secondary structures (FIGS. 6-16).

To test if these structures were capable of guiding the Lapis Cas protein to recognize a DNA target, the Lapis tracrRNA-like sequences (SEQ ID NOs: 114, 117, and 118) were synthesized as DNA sequences (Integrated DNA Technologies) with a suitable T7 polymerase initiation sequence and a 20 bp sequence, T1, CGCTAAAGAGGAAGAGGACA (SEQ ID NO: 121), for targeting a randomized PAM library as described previously (see PCT/US16/32073 filed May 12, 2016, PCT/US16/32028 filed May 12, 2016, incorporated in their entirety herein by reference (see Example 1)), being appended to the 5 prime end resulting in SEQ ID NOs: 122-124. The synthesized fragments were then PCR amplified adding on a promoter capable of directing T7 polymerase transcription and used as template for the production of guide RNAs containing the T1 spacer utilizing TranscriptAid T7 High Yield Transcription Kit (Thermo Fisher Scientific). Next, the resulting guide RNAs (SEQ ID NOs: 125-127) were complexed with Lapis Cas protein produced by in vitro translation and examined for their ability to support cleavage against a plasmid DNA 7 bp randomized PAM library as described previously (see PCT/US16/32073 filed May 12, 2016, PCT/US16/32028 filed May 12, 2016, incorporated in their entirety herein by reference (see Example 15)). Surprisingly, cleavage activity was detected with all 3 naturally occurring single guide RNAs and protospacer adjacent motif (PAM) sequences permitting cleavage recovered (Table 4) as described previously (see PCT/US16/32073 filed May 12, 2016, PCT/US16/32028 filed May 12, 2016, incorporated in their entirety herein by reference (see Examples 8 and 14)).

Taken together, this indicated that the putative tracrRNAs encoded in the Lactobacillus apis (Lapis) CRISPR-Cas locus, described herein (SEQ ID NOs: 110-120), with the addition of a 5 prime sequence containing homology to a target sequence that is adjacent to an appropriate PAM sequence, function as single naturally occurring CRISPR RNAs (snocrRNAs) engineered to direct Lapis Cas DNA target recognition Thus, presenting a novel in cis mechanism for the production of RNA species capable of directing Cas endonuclease target recognition that is different than the trans-encoding CRISPR RNA (tracrRNA) approach employed by Type II CRISPR-Cas systems (Deltcheva et al. (2011) Nature. 471:602-607, Makarova et al. (2011) Nature Reviews. 9:467-477, Chylinski et al. (2014) Nucleic Acids Research. 42:6091-6105, and Briner et al. (2014)).

Chimeric engineered guide RNAs capable of forming a guide RNA/Cas complex and directing the Lapis Cas protein to a DNA target sequence may be produced by adding a nucleotide sequence with homology to a DNA target sequence (also referred to as a variable targeting domain) 5 prime to the putative tracrRNAs described herein. Examples of such guide RNAs are listed in SEQ ID NOs: 128-138 where N may be any nucleotide.

TABLE 4
Position frequency matrix (PFM) and PAM consensus for Lactobacillus apis Cas protein.

| | 1 | 2 | 3 | 4 | 5 | 6 | 7 |
|---|---|---|---|---|---|---|---|
| G | 14.96% | 5.46% | 14.94% | 21.29%* | 6.11% | 11.39% | 16.57% |
| C | 54.87%* | 39.83%* | 14.85% | 35.61% | 42.81%* | 32.17% | 32.42% |
| A | 8.20% | 34.06%* | 69.34%* | 22.29% | 29.32%* | 43.04%* | 27.24%* |
| T | 21.97% | 20.65%* | 0.87% | 20.81% | 21.76%* | 13.40% | 23.76%* |
| Consensus | C | H | A | N | H | N | N |

Next, it was observed that the snocrRNAs while present individually were also associated with each CRISPR array (FIG. 5). Close examination of the repeat-spacer-repeat structure typical of a CRISPR array (Bhaya et al. (2011) and Wiedenheft et al. (2012)) revealed that the 5 prime end of the snocrRNAs (when transcribed in an anti-sense direction relative to the Lapis cas gene) formed the 5 prime most CRISPR repeat boundary of each array (FIG. 5, labels f, g, h, i, j, and k). This direct association with the CRISPR array suggests that the tracrRNA-like structures present in the snocrRNA are directly transcribed in cis with each CRISPR array (in an anti-sense orientation to the Lapis cas gene). Taken together, this further distinguishes the CRISPR-Cas system of Lactobacillus apis (Lapis) from Type II CRISPR-Cas sytems as it provides an in cis mechanism as opposed to a trans-encoded and activation model typified by the Type II CRISPR-Cas system (Deltcheva et al. (2011) Nature. 471:602-607, Makarova et al. (2011) Nature Reviews. 9:467-477, Chylinski et al. (2014) Nucleic Acids Research. 42:6091-6105, and Briner et al. (2014) Mol. Cell. 56:333-339).

### Example 3

### Transformation of Maize Immature Embryos

Transformation can be accomplished by various methods known to be effective in plants, including particle-mediated delivery, Agrobacterium-mediated transformation, PEG-mediated delivery, and electroporation.

a. Particle-Mediated Delivery

Transformation of maize immature embryos using particle delivery is performed as follows. Media recipes follow below.

The ears are husked and surface sterilized in 30% Clorox bleach plus 0.5% Micro detergent for 20 minutes, and rinsed two times with sterile water. The immature embryos are isolated and placed embryo axis side down (scutellum side up), 25 embryos per plate, on 560Y medium for 4 hours and then aligned within the 2.5-cm target zone in preparation for bombardment. Alternatively, isolated embryos are placed on 560L (Initiation medium) and placed in the dark at temperatures ranging from 26° C. to 37° C. for 8 to 24 hours prior to placing on 560Y for 4 hours at 26° C. prior to bombardment as described above.

Plasmids containing the double strand brake inducing agent and donor DNA are constructed using standard molecular biology techniques and co-bombarded with plasmids containing the developmental genes ODP2 (AP2 domain transcription factor ODP2 (Ovule development protein 2); US20090328252 A1) and Wushel (US2011/0167516).

The plasmids and DNA of interest are precipitated onto 0.6 μm (average diameter) gold pellets using a water-soluble cationic lipid transfection reagent as follows. DNA solution is prepared on ice using 1 μg of plasmid DNA and optionally other constructs for co-bombardment such as 50 ng (0.5 μl) of each plasmid containing the developmental genes ODP2 (AP2 domain transcription factor ODP2 (Ovule development protein 2); US20090328252 A1) and Wushel. To the pre-mixed DNA, 20 μl of prepared gold particles (15 mg/ml) and 5 μl of a water-soluble cationic lipid transfection reagent is added in water and mixed carefully. Gold particles are pelleted in a microfuge at 10,000 rpm for 1 min and supernatant is removed. The resulting pellet is carefully rinsed with 100 ml of 100% EtOH without resuspending the pellet and the EtOH rinse is carefully removed. 105 μl of 100% EtOH is added and the particles are resuspended by brief sonication. Then, 10 μl is spotted onto the center of each macrocarrier and allowed to dry about 2 minutes before bombardment.

Alternatively, the plasmids and DNA of interest are precipitated onto 1.1 μm (average diameter) tungsten pellets using a calcium chloride (CaCl₂)) precipitation procedure by mixing 100 μl prepared tungsten particles in water, 10 μl (1 μg) DNA in Tris EDTA buffer (1 μg total DNA), 100 μl 2.5 M CaC12, and 10 μl 0.1 M spermidine. Each reagent is added sequentially to the tungsten particle suspension, with mixing. The final mixture is sonicated briefly and allowed to incubate under constant vortexing for 10 minutes. After the precipitation period, the tubes are centrifuged briefly, liquid is removed, and the particles are washed with 500 ml 100% ethanol, followed by a 30 second centrifugation. Again, the liquid is removed, and 105 μl of 100% ethanol is added to the final tungsten particle pellet. For particle gun bombardment, the tungsten/DNA particles are briefly sonicated. 10 μl of the tungsten/DNA particles is spotted onto the center of each macrocarrier, after which the spotted particles are allowed to dry about 2 minutes before bombardment.

The sample plates are bombarded at level #4 with a Biorad Helium Gun. All samples receive a single shot at 450 PSI, with a total of ten aliquots taken from each tube of prepared particles/DNA.

Following bombardment, the embryos are incubated on 560P (maintenance medium) for 12 to 48 hours at temperatures ranging from 26 C to 37 C, and then placed at 26 C. After 5 to 7 days the embryos are transferred to 560R selection medium containing 3 mg/liter Bialaphos, and subcultured every 2 weeks at 26 C. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to a lighted culture room. Approximately 7-10 days later, developing plantlets are transferred to 272V hormone-free medium in tubes for 7-10 days until plantlets are well established. Plants are then transferred to inserts in flats (equivalent to a 2.5″ pot) containing potting soil and grown for 1 week in a growth chamber, subsequently grown an additional 1-2 weeks in the greenhouse, then transferred to Classic 600 pots (1.6 gallon) and grown to maturity. Plants are monitored and scored for transformation efficiency, and/or modification of regenerative capabilities.

Initiation medium (560L) comprises 4.0 g/l N6 basal salts (SIGMA C-1416), 1.0 ml/l Eriksson's Vitamin Mix (1000× SIGMA-1511), 0.5 mg/l thiamine HCl, 20.0 g/l sucrose, 1.0 mg/l 2,4-D, and 2.88 g/l L-proline (brought to volume with D-I H2O following adjustment to pH 5.8 with KOH); 2.0 g/l Gelrite (added after bringing to volume with D-I H2O); and 8.5 mg/l silver nitrate (added after sterilizing the medium and cooling to room temperature).

Maintenance medium (560P) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, 2.0 mg/l 2,4-D, and 0.69 g/l L-proline (brought to volume with D-I H₂O following adjustment to pH 5.8 with KOH); 3.0 g/l Gelrite (added after bringing to volume with D-I H2O); and 0.85 mg/l silver nitrate (added after sterilizing the medium and cooling to room temperature).

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D, and 2.88 g/l L-proline (brought to volume with D-I H2O following adjustment to pH 5.8 with KOH); 2.0 g/l Gelrite (added after bringing to volume with D-I H2O); and 8.5 mg/l silver nitrate (added after sterilizing the medium and cooling to room temperature).

Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-I H2O following adjustment to pH 5.8 with KOH); 3.0 g/l Gelrite (added after bringing to volume with D-I H2O); and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H2O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H2O after adjusting to pH 5.6); 3.0 g/l Gelrite (added after bringing to volume with D-I H2O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H2O), 0.1 g/l myo-inositol, and 40.0 g/l sucrose (brought to volume with polished D-I H2O after adjusting pH to 5.6); and 6 g/l bacto-agar (added after bringing to volume with polished D-I H2O), sterilized and cooled to 60° C.

b. Agrobacterium-Mediated Transformation

Agrobacterium-mediated transformation was performed essentially as described in Djukanovic et al. (2006) Plant Biotech J 4:345-57. Briefly, 10-12 day old immature embryos (0.8-2.5 mm in size) were dissected from sterilized kernels and placed into liquid medium (4.0 g/L N6 Basal Salts (Sigma C-1416), 1.0 ml/L Eriksson's Vitamin Mix (Sigma E-1511), 1.0 mg/L thiamine HCl, 1.5 mg/L 2, 4-D, 0.690 g/L L-proline, 68.5 g/L sucrose, 36.0 g/L glucose, pH 5.2). After embryo collection, the medium was replaced with 1 ml Agrobacterium at a concentration of 0.35-0.45 OD550. Maize embryos were incubated with Agrobacterium for 5 min at room temperature, then the mixture was poured onto a media plate containing 4.0 g/L N6 Basal Salts (Sigma C-1416), 1.0 ml/L Eriksson's Vitamin Mix (Sigma E-1511), 1.0 mg/L thiamine HCl, 1.5 mg/L 2, 4-D, 0.690 g/L L-proline, 30.0 g/L sucrose, 0.85 mg/L silver nitrate, 0.1 nM acetosyringone, and 3.0 g/L Gelrite, pH 5.8. Embryos were incubated axis down, in the dark for 3 days at 20° C., then incubated 4 days in the dark at 28° C., then transferred onto new media plates containing 4.0 g/L N6 Basal Salts (Sigma C-1416), 1.0 ml/L Eriksson's Vitamin Mix (Sigma E-1511), 1.0 mg/L thiamine HCl, 1.5 mg/L 2, 4-D, 0.69 g/L L-proline, 30.0 g/L sucrose, 0.5 g/L MES buffer, 0.85 mg/L silver nitrate, 3.0 mg/L Bialaphos, 100 mg/L carbenicillin, and 6.0 g/L agar, pH 5.8. Embryos were subcultured every three weeks until transgenic events were identified. Somatic embryogenesis was induced by transferring a small amount of tissue onto regeneration medium (4.3 g/L MS salts (Gibco 11117), 5.0 ml/L MS Vitamins Stock Solution, 100 mg/L myo-inositol, 0.1 μM ABA, 1 mg/L IAA, 0.5 mg/L zeatin, 60.0 g/L sucrose, 1.5 mg/L Bialaphos, 100 mg/L carbenicillin, 3.0 g/L Gelrite, pH 5.6) and incubation in the dark for two weeks at 28° C. All material with visible shoots and roots were transferred onto media containing 4.3 g/L MS salts (Gibco 11117), 5.0 ml/L MS Vitamins Stock Solution, 100 mg/L myo-inositol, 40.0 g/L sucrose, 1.5 g/L Gelrite, pH 5.6, and incubated under artificial light at 28° C. One week later, plantlets were moved into glass tubes containing the same medium and grown until they were sampled and/or transplanted into soil.

### Example 4

### Transient Expression of BBM Enhances Transformation

Parameters of the transformation protocol can be modified to ensure that the BBM activity is transient. One such method involves precipitating the BBM-containing plasmid in a manner that allows for transcription and expression, but precludes subsequent release of the DNA, for example, by using the chemical PEI. In one example, the BBM plasmid is precipitated onto gold particles with PEI, while the transgenic expression cassette (UBI::moPAT˜GFPm::PinII; moPAT is the maize optimized PAT gene) to be integrated is precipitated onto gold particles using the standard calcium chloride method.

Briefly, gold particles were coated with PEI as follows. First, the gold particles were washed. Thirty-five mg of gold particles, 1.0 in average diameter (A.S.I. #162-0010), were weighed out in a microcentrifuge tube, and 1.2 ml absolute EtOH was added and vortexed for one minute. The tube was incubated for 15 minutes at room temperature and then centrifuged at high speed using a microfuge for 15 minutes at 4° C. The supernatant was discarded and a fresh 1.2 ml aliquot of ethanol (EtOH) was added, vortexed for one minute, centrifuged for one minute, and the supernatant again discarded (this is repeated twice). A fresh 1.2 ml aliquot of EtOH was added, and this suspension (gold particles in EtOH) was stored at −20° C. for weeks. To coat particles with polyethylimine (PEI; Sigma #P3143), 250 μl of the washed gold particle/EtOH mix was centrifuged and the EtOH discarded. The particles were washed once in 100 μl ddH2O to remove residual ethanol, 250 μl of 0.25 mM PEI was added, followed by a pulse-sonication to suspend the particles and then the tube was plunged into a dry ice/EtOH bath to flash-freeze the suspension, which was then lyophilized overnight. At this point, dry, coated particles could be stored at −80° C. for at least 3 weeks. Before use, the particles were rinsed 3 times with 250 μl aliquots of 2.5 mM HEPES buffer, pH 7.1, with 1× pulse-sonication, and then a quick vortex before each centrifugation. The particles were then suspended in a final volume of 250 μl HEPES buffer. A 25 μl aliquot of the particles was added to fresh tubes before attaching DNA. To attach uncoated DNA, the particles were pulse-sonicated, then 1 μg of DNA (in 5 μl water) was added, followed by mixing by pipetting up and down a few times with a Pipetteman and incubated for 10 minutes. The particles were spun briefly (i.e. 10 seconds), the supernatant removed, and 60 μl EtOH added. The particles with PEI-precipitated DNA-1 were washed twice in 60 μl of EtOH. The particles were centrifuged, the supernatant discarded, and the particles were resuspended in 45 μl water. To attach the second DNA (DNA-2), precipitation using a water-soluble cationic lipid transfection reagent was used. The 45 μl of particles/DNA-1 suspension was briefly sonicated, and then 5 μl of 100 ng/μl of DNA-2 and 2.5 μl of the water-soluble cationic lipid transfection reagent were added. The solution was placed on a rotary shaker for 10 minutes, centrifuged at 10,000 g for 1 minute. The supernatant was removed, and the particles resuspended in 60 μl of EtOH. The solution was spotted onto macrocarriers and the gold particles onto which DNA-1 and DNA-2 had been sequentially attached were delivered into scutellar cells of 10 DAP Hi-II immature embryos using a standard protocol for the PDS-1000. For this experiment, the DNA-1 plasmid contained a UBI::RFP::pinII expression cassette, and DNA-2 contained a UBI::CFP::pinII expression cassette. Two days after bombardment, transient expression of both the CFP and RFP fluorescent markers was observed as numerous red & blue cells on the surface of the immature embryo. The embryos were then placed on non-selective culture medium and allowed to grow for 3 weeks before scoring for stable colonies. After this 3-week period, 10 multicellular, stably-expressing blue colonies were observed, in comparison to only one red colony. This demonstrated that PEI-precipitation could be used to effectively introduce DNA for transient expression while dramatically reducing integration of the PEI-introduced DNA and thus reducing the recovery of RFP-expressing transgenic events. In this manner, PEI-precipitation can be used to deliver transient expression of BBM and/or WUS2.

For example, the particles are first coated with UBI::BBM::pinII using PEI, then coated with UBI::moPAT-YFP using a water-soluble cationic lipid transfection reagent, and then bombarded into scutellar cells on the surface of immature embryos. PEI-mediated precipitation results in a high frequency of transiently expressing cells on the surface of the immature embryo and extremely low frequencies of recovery of stable transformants Thus, it is expected that the PEI-precipitated BBM cassette expresses transiently and stimulates a burst of embryogenic growth on the bombarded surface of the tissue (i.e. the scutellar surface), but this plasmid will not integrate. The PAT-GFP plasmid released from the Ca++/gold particles is expected to integrate and express the selectable marker at a frequency that results in substantially improved recovery of transgenic events. As a control treatment, PEI-precipitated particles containing a UBI::GUS::pinII (instead of BBM) are mixed with the PAT-GFP/Ca++ particles. Immature embryos from both treatments are moved onto culture medium containing 3 mg/l bialaphos. After 6-8 weeks, it is expected that GFP+, bialaphos-resistant calli will be observed in the PEI/BBM treatment at a much higher frequency relative to the control treatment (PEI/GUS).

As an alternative method, the BBM plasmid is precipitated onto gold particles with PEI, and then introduced into scutellar cells on the surface of immature embryos, and subsequent transient expression of the BBM gene elicits a rapid proliferation of embryogenic growth. During this period of induced growth, the explants are treated with Agrobacterium using standard methods for maize (see Example 1), with T-DNA delivery into the cell introducing a transgenic expression cassette such as UBI::moPAT˜GFPm::pinII. After co-cultivation, explants are allowed to recover on normal culture medium, and then are moved onto culture medium containing 3 mg/l bialaphos. After 6-8 weeks, it is expected that GFP+, bialaphos-resistant calli will be observed in the PEI/BBM treatment at a much higher frequency relative to the control treatment (PEI/GUS).

It may be desirable to “kick start” callus growth by transiently expressing the BBM and/or WUS2 polynucleotide products. This can be done by delivering BBM and WUS2 5′-capped polyadenylated RNA, expression cassettes containing BBM and WUS2 DNA, or BBM and/or WUS2 proteins. All of these molecules can be delivered using a biolistics particle gun. For example 5′-capped polyadenylated BBM and/or WUS2 RNA can easily be made in vitro using Ambion's mMessage mMachine kit. RNA is co-delivered along with DNA containing a polynucleotide of interest and a marker used for selection/screening such as Ubi::moPAT˜GFPm::PinII. It is expected that the cells receiving the RNA will immediately begin dividing more rapidly and a large portion of these will have integrated the agronomic gene. These events can further be validated as being transgenic clonal colonies because they will also express the PAT-GFP fusion protein (and thus will display green fluorescence under appropriate illumination). Plants regenerated from these embryos can then be screened for the presence of the polynucleotide of interest.

1. A guide RNA/Cas endonuclease complex comprising at least one guide RNA and a Cas endonuclease, wherein said Cas endonuclease comprises a first nuclease domain of SEQ ID NO: 88, or a functional fragment or functional variant of SEQ ID NO:88, and a second nuclease domain comprising at least one nuclease subdomain selected from the group consisting of SEQ ID NO: 90, SEQ ID NO: 92 and SEQ ID NO: 94, or a functional fragment or functional variant of said second nuclease domain, wherein said guide RNA is a chimeric engineered guide RNA, wherein said guide RNA/Cas endonuclease complex is capable of recognizing, binding to, and optionally nicking, cleaving, or covalently attaching to all or part of a target sequence.

2. The guide RNA/Cas endonuclease complex of claim 1, wherein said Cas endonuclease has at least 80% sequence identity to SEQ ID NO: 1 and wherein said Cas endonuclease is not a Type II Cas9 endonuclease.

3. The guide RNA/Cas endonuclease complex of claim 1 or claim 2, comprising at least one chimeric engineered guide RNA comprising a variable targeting domain that can recognize a target DNA in a eukaryotic cell.

4. The guide RNA/Cas endonuclease complex of claim 1 or claim 2, wherein said target sequence is located in the genome of a prokaryotic or eukaryotic cell.

5. A method for modifying a target site in the genome of a cell, the method comprising introducing into said cell at least one chimeric engineered guide RNA, and a Cas endonuclease comprising a first nuclease domain of SEQ ID NO: 88, and a second nuclease domain comprising at least one nuclease sub-domain selected from the group consisting of SEQ ID NO: 90, SEQ ID NO: 92 and SEQ ID NO: 94, wherein said chimeric engineered guide RNA and Cas endonuclease can form a complex that is capable of recognizing, binding to, and optionally nicking, cleaving, or covalently attaching to all or part of said target site; further comprising identifying at least one cell that has a modification at said target, wherein the modification at said target site is selected from the group consisting of (i) a replacement of at least one nucleotide, (ii) a deletion of at least one nucleotide, (iii) an insertion of at least one nucleotide, and (iv) any combination of (i)-(iii).

6. (canceled)

7. A method for editing a nucleotide sequence in the genome of a cell, the method comprising introducing into said cell at least one polynucleotide modification template, at least one chimeric engineered guide RNA, and a Cas endonuclease comprising a first nuclease domain of SEQ ID NO: 88, and a second nuclease domain comprising at least one nuclease sub-domain selected from the group consisting of SEQ ID NO: 90, SEQ ID NO: 92 and SEQ ID NO: 94, wherein said polynucleotide modification template comprises at least one nucleotide modification of said nucleotide sequence, wherein said chimeric engineered guide RNA and Cas endonuclease can form a complex that is capable of recognizing, binding to, and optionally nicking, cleaving, or covalently attaching to all or part of said target site.

8. A method for modifying a target site in the genome of a cell, the method comprising providing to said cell at least one chimeric engineered guide RNA, at least one donor DNA, and a Cas endonuclease comprising a first nuclease domain of SEQ ID NO: 88, and a second nuclease domain comprising at least one nuclease sub-domain selected from the group consisting of SEQ ID NO: 90, SEQ ID NO: 92 and SEQ ID NO: 94, wherein said at least one chimeric engineered guide RNA and Cas endonuclease can form a complex that is capable of recognizing, binding to, and optionally nicking, cleaving, or covalently attaching to all or part of said target site, wherein said donor DNA comprises a polynucleotide of interest; further comprising identifying at least one cell that said polynucleotide of interest integrated in or near said target site.

9. (canceled)

10. (canceled)

11. The method of any one of claims 5, 7, or 8, wherein the cell is selected from the group consisting of a mammalian, human cell, non-human cell, animal cell, bacterial cell, fungal cell, insect cell, yeast cell, non-conventional yeast cell, and a plant cell.

12. (canceled)

13. The method of claim 11 wherein the plant cell is selected from the group consisting of maize, rice, sorghum, rye, barley, wheat, millet, oats, sugarcane, turfgrass, switchgrass, soybean, canola, alfalfa, sunflower, cotton, tobacco, peanut, potato, Arabidopsis, and safflower cell.

14. (canceled)

15. (canceled)

16. (canceled)

17. A recombinant DNA polynucleotide comprising a promoter operably linked to a plant-optimized polynucleotide encoding a Cas endonuclease, wherein said Cas endonuclease comprises a first nuclease domain of SEQ ID NO: 88, and a second nuclease domain comprising at least one nuclease sub-domain selected from the group consisting of SEQ ID NO: 90, SEQ ID NO: 92 and SEQ ID NO: 94.

18. A kit for binding, cleaving or nicking a target sequence in a prokaryotic or eukaryotic cell or organism, said kit comprising a guide polynucleotide specific for said target sequence, and a Cas endonuclease or a polynucleotide encoding said Cas endonuclease, wherein said Cas endonuclease comprises a first nuclease domain of SEQ ID NO: 88, and a second nuclease domain comprising at least one nuclease sub-domain selected from the group consisting of SEQ ID NO: 90, SEQ ID NO: 92 and SEQ ID NO: 94, wherein said guide polynucleotide is capable of forming a guide polynucleotide/Cas endonuclease complex, wherein said complex can recognize, bind to, and optionally nick or cleave said target sequence.

19. A chimeric engineered guide RNA capable of forming a guide RNA/Cas endonuclease complex that can recognize, bind to, and optionally nick or cleave a target sequence, wherein said guide RNA is selected from the group consisting of SEQ ID NOs: 128-138.
